(19) United States
(12) Patent Application Publication
(10) Pub. No.: US 2024/0115414 A1
(43) Pub. Date: Apr. 11, 2024

(54) ORAL APPLIANCE FOR THE TREATMENT OF SLEEP APNEA

(71) Applicant: Achaemenid, LLC, Stratford, CT (US)

(72) Inventor: Reza RADMAND, Boston, MA (US)

(21) Appl. No.: 18/391,482

(22) Filed: Dec. 20, 2023

Related U.S. Application Data
(63) Continuation-in-part of application No. 18/209,298, filed on Jun. 13, 2023, now abandoned.
(60) Provisional application No. 63/411,766, filed on Sep. 30, 2022.
(60) Provisional application No. 63/392,959, filed on Jul. 28, 2022.
(60) Provisional application No. 63/390,923, filed on Jul. 20, 2022.
(60) Provisional application No. 63/389,969, filed on Jul. 18, 2022.

(51) Int. Cl. A61F 5/56; A61N 1/02; A61N 1/36

(52) U.S. Cl. CPC A61F 5/566; A61N 1/025; A61N 1/3601

(57) ABSTRACT

An oral appliance for treating sleep apnea in a user includes a mouthpiece configured for being positioned in an oral cavity of the user, a processor, and at least one sensor coupled to the mouthpiece. According to an aspect, the processor may be configured to detect a condition of a user based on data generated by the at least one sensor. Additionally, the sensor may include at least one of a sound measuring device, an inertial measurement unit, a pulse oximeter, or an electroencephalogram.

### CROSS-REFERENCE TO RELATED APPLICATIONS

This application is a continuation-in-part of U.S. patent application Ser. No. 18/209,298, filed Jun. 13, 2023, and entitled “Oral Appliance for the Treatment of Sleep Apnea”, which is hereby incorporated by reference herein, which in turn claims priority to each of: claims the benefit of: (i) U.S. Provisional Patent Application No. 63/411,766 filed on Sep. 30, 2022; (ii) U.S. Provisional Patent Application No. 63/392,959 filed on Jul. 28, 2022; (iii) U.S. Provisional Patent Application No. 63/390,923 filed on Jul. 20, 2022; and (iv) U.S. Provisional Patent Application No. 63/389,969 filed on Jul. 18, 2022, the entire contents of each of which being incorporated by reference herein.

### BACKGROUND

Sleep apnea is a common medical condition during which a person experiences one or more pauses in breathing, and in some instances, experiences shallow breaths during sleep. While there are several types of sleep apnea, the most common type is obstructive sleep apnea. In this medical condition, one or more of the person's throat muscles relax during sleep causing surrounding tissues in the posterior portions of the mouth, nose and throat to collapse, thereby creating a pharyngeal obstruction that can block the upper airway. Persons suffering from obstructive sleep apnea have inadequate oxygen exchange during sleep, which can lead to daytime fatigue, lack of concentration and mood changes. Left untreated, obstructive sleep apnea can have a significant impact on a person's health, often leading to cardiovascular, stroke and metabolic disorders.

To reduce this risk, various nonsurgical approaches have been employed. One such nonsurgical approach includes using standardized oral appliances to incrementally advance and/or protrude the mandible (lower jaw) relative to the maxilla (upper jaw). These standardized oral appliances, commonly referred to as a mandibular advancement device, (“MAD”), typically include upper and lower dental trays, whereby the lower dental tray is designed to advance the mandible, and hence, move the tongue forward to increase the space in the posterior part of the throat and the oropharynx, which in turn may serve to increase the flow of air during sleep. The distance (degree of advancement) required to protrude and/or reposition the mandible may be, at least in part, dependent on the severity of the individual's obstructive sleep apnea, as well as psychological variables among the users. A disadvantage of using these standard oral appliances is that they may not sufficiently provide for and/or address individualized anatomical variances, such as difference in dental arches, dentition alignment and/or jaw flexibility. Another disadvantage is that in instances where the degree of advancement is excessive, the appliance may lead to long-term temporomandibular joint (“TMJ”) disorders, muscular aggravation, dentition discomfort and/or myofascial disorders. As a result, use of these standard appliances has an approximate compliance rate of 75% over a 2-year period. For a detailed study of compliance with use of MAD, see Non-CPAP therapies in obstructive sleep apnea: mandibular advancement device therapy, see Eur Respir J 2012; 39: 1241-1247, which is incorporated by reference in its entirety. Thus, such oral appliances may not treat obstructive sleep apnea in a manner that prevents and/or limits impacts on a person's health.

In view of the disadvantages associated with currently available methods and devices for treating obstructive sleep apnea, there is a need for a device and method that treats obstructive sleep apnea while storing patient behavior and/or medical data relating to a user's oxygen saturation, breathing pattern, snoring pattern and/or clenching/grinding behaviors, that can assist medical providers in the design, improvement and/or modification of specialized treatment measures for individual patients. Further, there is a need for a device and method that treats obstructive sleep apnea in a single removable oral appliance and prevents and/or limits long-term TMJ disorders, muscular aggravation and/or myofascial disorders that may occur with continued use of currently available appliances.

### BRIEF DESCRIPTION

According to an aspect of the present disclosure, an exemplary embodiment of an oral appliance includes a mouthpiece configured to be positioned in an oral cavity of a user, and an electronic assembly coupled to the mouthpiece. The mouthpiece includes a housing and a cover. The housing has a first portion configured to engage an occlusal surface of a user's dentition, and an opposite second portion defining an inner chamber. The cover is secured to the second portion of the housing to seal the inner chamber from an external environment. The electronic assembly includes an oxygen sensor configured to be oriented toward soft tissue of the user's mouth to determine an oxygen level of the user.

According to another aspect of the present disclosure, an exemplary embodiment of an oral appliance for treating sleep apnea in a user includes a mouthpiece configured to be positioned over upper or lower dentition of the user, a housing coupled to the mouthpiece and defining an inner chamber, and one or more sensors positioned in the inner chamber of the housing. One such sensor includes a reflectance pulse oximeter having a light emitting diode (LED) configured to transmit light. The reflectance pulse oximeter is configured to collect peripheral oxygen saturation (SpO2) and photoplethysmography (PPG) data via the light.

According to yet another aspect of the present disclosure, an exemplary embodiment of an oral appliance for treating sleep apnea in a user includes a mouthpiece configured to be positioned over upper or lower dentition of the user, a housing, and a printed circuit board assembly. The mouthpiece includes an anterior wall configured to cover facial surfaces of the user's dentition, a posterior wall configured to cover lingual surfaces of the user's dentition, and a transverse wall interconnecting the anterior wall and the posterior wall and configured to cover an occlusal surface of the user's dentition. The housing includes a posterior portion coupled to the anterior wall of the mouthpiece and an anterior portion coupled to the posterior portion. The posterior portion has an optical window fabricated from a light-transmissive material. The posterior portion and the anterior portion collectively define an inner chamber of the housing. The printed circuit board assembly is positioned in the inner chamber of the housing and includes a printed circuit board, a pair of barriers coupled to the printed circuit board, and a reflectance pulse oximeter coupled to an in electrical communication with the printed circuit board. The pair of barriers define a space therebetween, and the reflectance pulse oximeter includes a light emitting diode (LED) positioned in the space and adjacent the optical window of the housing. The LED is configured to transmit red light and infrared light. The reflectance pulse oximeter is configured to determine an oxygen saturation of hemoglobin of the user via the red light and the infrared light.

According to yet another aspect of the present disclosure, an oral appliance is provided that includes a mouthpiece configured to be positioned in an oral cavity of a user, a housing defining an inner chamber, and an oxygen sensor positioned in the inner chamber of the housing. The housing has a posterior portion and an anterior portion. The posterior portion is coupled to the mouthpiece and has an optical window. The oxygen sensor is configured to determine an oxygen level of the user.

According to yet another aspect of the present disclosure, an oral appliance is provided that includes a mouthpiece configured to be positioned in an oral cavity of a user and a sound measuring device coupled to the mouthpiece. The sound measuring device is configured to detect at least one of the user's normal breathing, changes in the user's breath sound intensity, or the user's snore sound quality.

According to yet another aspect of the present disclosure, an oral noise monitor is provided that includes a mouthpiece configured to be positioned in an oral cavity of a user, a cannula coupled to the mouthpiece, and a sound measuring device adjacent the cannula. The sound measuring device is configured to detect at least one of the user's normal breathing, changes in the user's breath sound intensity, or the user's snore sound quality.

### BRIEF DESCRIPTION OF THE FIGURES

A more particular description will be rendered by reference to specific embodiments thereof that are illustrated in the appended drawings. Understanding that these drawings depict only typical embodiments thereof and are not therefore to be considered to be limiting of its scope, exemplary embodiments will be described and explained with additional specificity and detail through the use of the accompanying drawings in which:

FIG. 1 is a perspective view of an exemplary oral appliance in accordance with aspects of the present disclosure;

FIG. 2 is a plan view of the oral appliance of FIG. 1

FIG. 3 is a plan view of the oral appliance of FIG. 1, with a cross-section taken along a horizontal plane of a housing assembly of the oral appliance;

FIG. 4 is a posterior view, with a posterior housing half removed, illustrating internal electrical components of a housing assembly of the oral appliance of FIG. 1;

FIG. 5 is an anterior view, with an anterior housing half removed, illustrating the internal electronic components of the housing assembly of FIG. 4;

FIG. 6 is a side, cutaway view of the housing assembly of the oral appliance of FIG. 1;

FIG. 7 is a plan view illustrating the internal electronic components of FIG. 6;

FIG. 8A is an anterior view of the internal electronic components of FIG. 6;

FIG. 8B is a top view of the internal electronic components;

FIG. 8C is a posterior view of the internal electronic components;

FIG. 9 is a schematic illustration of components of an oxygen sensor positioned between isolation barriers of the oral appliance of FIG. 1;

FIG. 10 is a side, perspective view illustrating another aspect of an oral appliance including a top mouthpiece, a bottom mouthpiece, and a strut assembly;

FIG. 11 is a plan view illustrating internal components of the top mouthpiece of the oral appliance of FIG. 10;

FIG. 12A is a plan view illustrating a housing of the top mouthpiece of FIG. 11;

FIG. 12B is a side, perspective view illustrating the housing of FIG. 12A;

FIG. 12C is a side, perspective view illustrating a cover of the top mouthpiece of FIG. 10;

FIG. 13 is a perspective view illustrating the top mouthpiece of the oral appliance of FIG. 10 positioned on a user's upper dentition;

FIG. 14 is a side, perspective view illustrating the oral appliance of FIG. 10 positioned on the upper dentition of the user;

FIGS. 15A-15H illustrate various aspects of an oral appliance, or mouth piece, with a sound measuring device;

FIGS. 16A-16C are charts illustrating various aspects of data generated by a sound measuring device;

FIG. 17 is a chart that depicts data relating to snoring and normal breathing sounds, collected by a sound measuring device, according to an aspect of the present disclosure;

FIG. 18 is a chart that depicts data relating to snoring and normal breathing sounds, collected by a sound measuring device, according to an aspect of the present disclosure;

FIG. 19 is a chart that depicts data relating to normal breathing sound, collected by a sound measuring device, according to an aspect of the present disclosure;

FIG. 20 is a chart that depicts data relating to normal breathing sound, collected by a sound measuring device, according to an aspect of the present disclosure;

FIG. 21 is a chart that depicts data relating to snoring and normal breathing sounds, collected by a sound measuring device, according to an aspect of the present disclosure;

FIG. 22 is a chart that depicts data relating to normal breathing sound, collected by a sound measuring device, according to an aspect of the present disclosure;

FIG. 23 is a chart that depicts data relating to snoring sounds, collected by a sound measuring device, according to an aspect of the present disclosure;

FIG. 24 is a chart that depicts data relating to snoring sounds, collected by a sound measuring device, according to an aspect of the present disclosure;

FIG. 25 is a chart that depicts data relating to normal breathing sound, collected by a sound measuring device, according to an aspect of the present disclosure;

FIG. 26A are two charts illustrating data collected by additional sensors such as an accelerometer and a gyroscope of an oral appliance of the present disclosure with a patient with an open mouth and an inertial measuring unit (IMU) intra-orally coupled to the oral appliance;

FIG. 26B are two charts illustrating data collected by an accelerometer and a gyroscope of an oral appliance of the present disclosure with a patient with an open mouth and an IMU extra-orally coupled to the patient's chin;

FIG. 27A are two charts illustrating data collected by an accelerometer and a gyroscope of an oral appliance of the present disclosure with a patient grinding their teeth in a side-to-side motion and an IMU intra-orally coupled to the oral appliance;

FIG. 27B are two charts illustrating data collected by an accelerometer and a gyroscope of an oral appliance of the present disclosure with a patient grinding their teeth in a side-to-side motion and an IMU extra-orally coupled to the patient's chin;

FIG. 28A are two charts illustrating data collected by an accelerometer and a gyroscope of an oral appliance of the present disclosure with a patient swallowing and an IMU intra-orally coupled to the oral appliance;

FIG. 28B are two charts illustrating data collected by an accelerometer and a gyroscope of an oral appliance of the present disclosure with a patient swallowing and an IMU extra-orally coupled to the patient's chin;

FIGS. 29A-29H are charts illustrating various aspects of data generated by an IMU;

FIG. 30 is a perspective view illustrating yet another embodiment of an oral appliance including a sensor assembly;

FIG. 31 is a partial, perspective view illustrating the sensor assembly of FIG. 30 with a charging coil thereof removed;

FIG. 32 is a partial, perspective view illustrating the sensor assembly of FIG. 30 with the charging coil and a spacer thereof removed;

FIG. 33 is another partial, perspective view illustrating a printed circuit board and a sensor of the sensor assembly of FIG. 32 positioned in the housing;

FIG. 34 is a bottom, perspective view illustrating an oral appliance including an electromyogram sensor according to another embodiment of the present disclosure;

FIG. 35 is a chart illustrating electromyogram data collected during tongue muscle stimulation and while a user's eyes are moved between opened and closed configurations; and

FIG. 36 is a chart illustrating the difference between electromyogram data from tongue muscle activation versus electromyogram data in the absence of tongue muscle activation.

Various features, aspects, and advantages of the embodiments will become more apparent from the following detailed description, along with the accompanying figures in which like numerals represent like components throughout the figures and text. The various described features are not necessarily drawn to scale but are drawn to emphasize specific features relevant to some embodiments.

### DETAILED DESCRIPTION

Reference will now be made in detail to various embodiments. Each example is provided by way of explanation and is not meant as a limitation and does not constitute a definition of all possible embodiments.

For purposes of illustrating features of the embodiments, embodiments will now be introduced and referenced throughout the disclosure. Those skilled in the art will recognize that this example is illustrative and not limiting and is provided purely for explanatory purposes.

As used herein, “coupled” may refer to any manner of assembling or connecting two components together in any suitable manner, such as, by way of example only: attaching directly or indirectly (e.g., attached to a surface), disposing on, disposing within, disposing substantially within, formed with, embedding within, embedded substantially within, etc. “Coupled” may further include fixedly attaching two components (such as by using a screw or embedding a first component into a second component during a manufacturing process), but does not so require. That is, two components may be coupled temporarily simply by being in physical contact with one another.

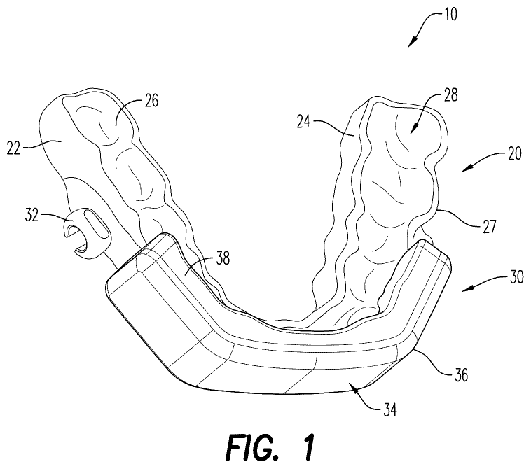

In an embodiment, and with particular reference to FIGS. 1-6, an oral appliance 10 for treatment of sleep apnea in a user is provided. The oral appliance 10 generally includes a mouthpiece 20 and a housing assembly 30 coupled to the mouthpiece 20. In an embodiment, the mouthpiece 20 is “customizable,” that is, customized to the individual user's mouth in such a manner that it provides for a comfortable fit over and around surfaces of the user's hard (teeth/dentition) and/or soft tissues (general mouth structure, including gingiva). When customized, the mouthpiece 20 may fit over temporary, permanent, primary natural and/or artificial lower or upper dentition of adult and/or child users. The mouthpiece 20 may be configured to receive a removable denture of the user. According to an aspect, the mouthpiece 20 is fabricated over the upper jaw or lower jaw with partial or complete absence of dentition. When customized, the mouthpiece 20 can be formed of any suitable self-conforming material that may be adaptable to variances and/or changes in mouth structure, or through use of a dental impression of the individual user's dentition, as would be understood by a person having ordinary skill in the art. In other words, a mandibular impression and/or a dental impression can be taken, whereby a negative imprint of the user's hard and/or soft tissues are used to create a positive reproduction (or cast) customized for the user. In aspects, the mandibular impression and/or dental impression may be a digital dental impression taken with 3D scanning technology or other suitable technologies.

The types of materials selected to form the mouthpiece 20 would be known to one of ordinary skill in the art and includes polymers, thermoplastics, acrylics, silicone, rubber, metal wires or any other material that can be used to form the mouthpiece 20 conformed to the user's dentition. In an embodiment, the materials are medical-grade, latex-free, BPA-free and any other material known to minimize patient health risks. According to an aspect, the mouthpiece 20 may be formed from the impression made in a suitable impression material, such as, for example, alginate, polysulfide, polyvinyl siloxane, silicone, or the like. The mouthpiece material may also be selected, particularly from polymers, for its ability to have a pharmaceutical compound incorporated within the structural matrix.

The mouthpiece 20 includes an anterior wall 22 configured to cover facial surfaces of a user's dentition, a posterior wall 24 configured to cover lingual surfaces of the user's dentition, and a transverse wall 26 interconnecting the anterior wall 22 and the posterior wall 24 and configured to cover an occlusal surface of the user's dentition. The mouthpiece 20 defines a central channel 28 bounded by the anterior wall 22, the posterior wall 24, and the transverse wall 26. The central channel 28 is configured to be positioned over one or more of the user's dentition such that the mouthpiece 20 is secured thereon. When the mouthpiece 20 is in use, the central channel 28 may receive the user's dentition and may extend over and/or cover occlusal or bite surfaces of the user's dentition. The posterior wall 24 of the mouthpiece 20 extends between the user's dentition and the user's tongue. In an embodiment, the anterior wall 22 of the mouthpiece 20 is configured to extend between the user's dentition and the user's cheek.

The mouthpiece 20 may include a socket 32 coupled to the anterior wall 22 of the mouthpiece 20. The socket 32 is configured to couple to a strut assembly, such as, for example, one or more of the strut assemblies disclosed in U.S. patent application Ser. No. 17/737,470 filed May 5, 2022, the entire contents of which are incorporated by reference herein. The oral appliance 10, in combination with the strut assemblies, may be configured to position the bottom jaw of a user in a direction that is forward from the natural position of the user's jaw to aid in the treatment of sleep apnea in the user. The best jaw position for the user may be determined by a dentist and prescribed to a lab that is tasked with fabricating the oral appliance 10.

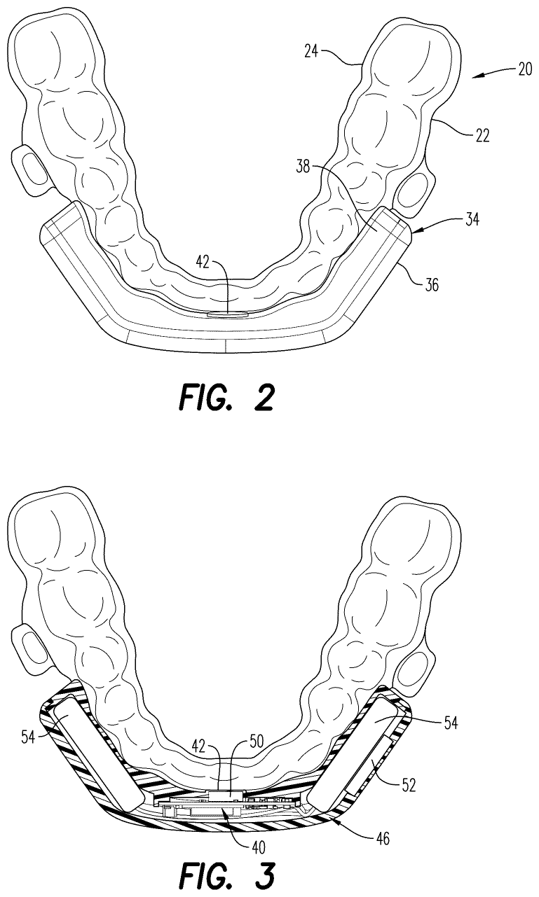

The housing assembly 30 of the oral appliance 10 generally includes a housing 34 and a printed circuit board assembly 40 (FIGS. 2 and 3) positioned within the housing 34. The housing 34 has an anterior portion 36, and a posterior portion 38 coupled to the anterior wall 22 of the mouthpiece 20. The anterior portion 36 and the posterior portion 38 may be monolithically formed with one another to form the housing 34, and in other aspects the anterior portion 36 and the posterior portion 38 may be separate components that are otherwise attached to one another. The posterior portion 38 of the housing 34 conforms to and is coupled to the anterior wall 22 of the mouthpiece 20 using any suitable fastening engagement, such as, for example, adhesives, fasteners, heat treatment, or the like. In other aspects, the posterior portion 38 of the housing 34 may be monolithically formed with the mouthpiece 20.

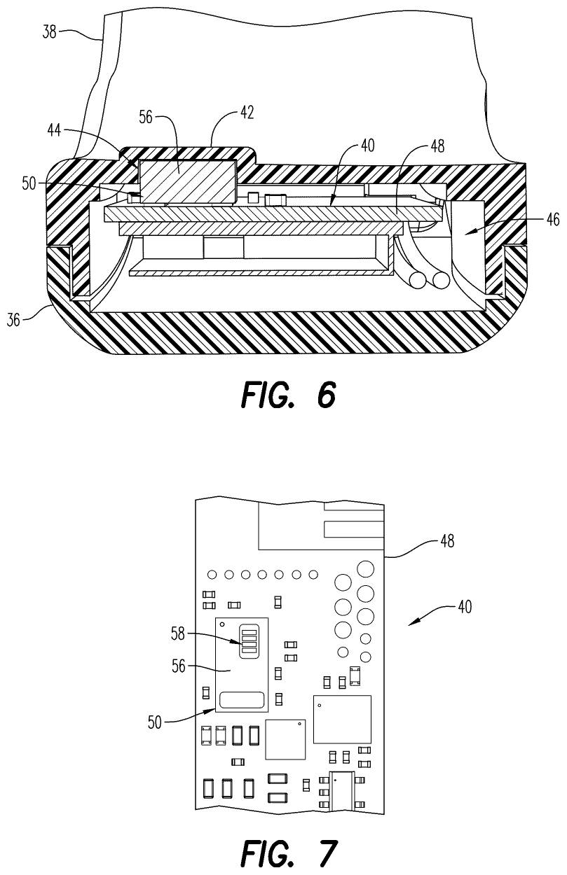

The posterior portion 38 protrudes anteriorly away from the mouthpiece 20 and perpendicularly away from a gingival edge 27 of the mouthpiece 20. The posterior portion 38 may be fabricated from a light-transmissive material, such as, for example, polycarbonate, acrylic, polyethylene terephthalate, amorphous copolyester (modified forms of polyester, such as combinations of diacids and diols), polyvinyl chloride, liquid silicone rubber, polyethylene, styrene methyl methacrylate, or the like. In aspects and as illustrated in FIG. 3, only a portion of the posterior portion 38 may be light-transmissive, such as, for example, an optical window 42 that is positioned at a midline of the posterior portion 38. With reference to FIG. 6, the optical window 42 is monolithically formed with the posterior portion 38 and may also protrude posteriorly from the posterior portion 38 to define a recess 44 therein for receiving an optical assembly 50 therein, as will be described in more detail below. As shown herein, the material forming the posterior portion 38 differs from the material forming the anterior portion 36.

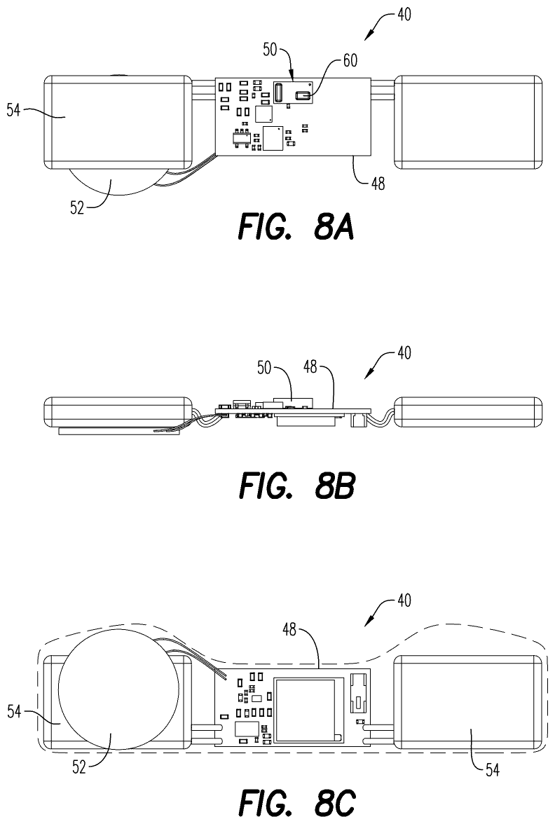

With particular reference to FIGS. 8A-C, the housing 34 defines an inner chamber 46 therein that extends along a length of the housing 34 for housing the printed circuit board assembly 40 therein. The printed circuit board assembly 40 includes a printed circuit board 48, the optical assembly 50, and in some aspects, a microprocessor (not labeled) and other suitable electronic components. For example, the printed circuit board assembly 40 may further include a wireless power charging coil 52 positioned at a first end portion of the printed circuit board 48 and one or more batteries 54, such as, for example, a lithium-polymer battery (LiPo) rechargeable batteries, positioned at the first end portion and a second end portion of the printed circuit board 48.

The optical assembly 50 is positioned adjacent the optical window 42 of the housing 34 and includes an optical housing 56 supported on a posterior side of the printed circuit board 48 and an oxygen sensor 60 supported in the optical housing 56 and electrically coupled to the microprocessor of the printed circuit board 48. In aspects, the optical assembly 50 may be supported in the recess 44 (FIG. 6) defined by the optical window 42 such that the optical assembly 50 is oriented on a sagittal plane of the user's oral cavity and towards buccal gingiva of the user when the oral appliance 10 is worn by the user.

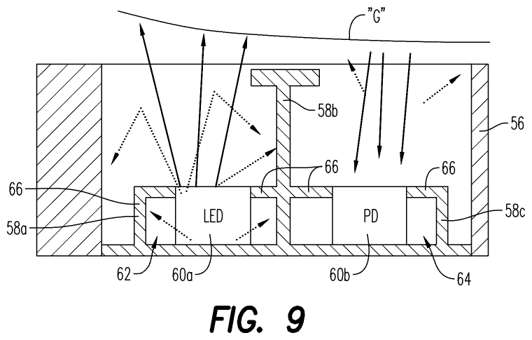

With reference to FIGS. 7 and 9, the optical housing 56 may include a plurality of isolation baffles or barriers 58 coupled to the printed circuit board 48. The barriers 58 are arranged in parallel relation with one another. Adjacent barriers, such as, for example, barriers 58a, 58b, define a first space 62 therebetween in which one or more components of the oxygen sensor 60 are positioned. The oxygen sensor 60 may be a reflectance pulse oximeter configured to monitor/sense the oxygen saturation of a user by analyzing the change in color of the user's blood. The reflectance pulse oximeter 60 may measure the pulse rate of the user, typically in beats per minute, based on variations and/or deviations in the user's oxygen saturation. An exemplary pulse oximeter may use light-based technology to sense the actual oxygen saturation of hemoglobin of the user. According to an aspect, the pulse oximeter 60 includes light emitting diodes (LEDs) 60a and photodiodes (PD) 60b. The LED 60a is configured to transmit red and infrared lights to vascular surfaces of the user's gingiva “G” and the photodiodes 60b receive the light reflected from the gingiva “G” to determine changes in oxygen level in the user's gingiva “G” (e.g., the alveolar mucosa).

The barriers 58 provide optical isolation between the LED 60a and the detection photodiode 60b to improve a signal-to noise ratio through tissue. For example, as shown in FIG. 9, the LED 60a may be received in the first space 62 between adjacent barriers 58a, 58b, and the photodiode 60b may be received in a second space 64 defined between adjacent barriers 58b, 58c. Each of the isolation barriers 58 may include a horizontal wall 66 extending perpendicularly therefrom to prevent laterally-projecting light from escaping the spaces 62, 64 between the barriers 58. In this way, the barriers 58 are configured to isolate the light projecting from the LEDs 60a. Moreover, the optical reflections reject both dc and ac ambient light interference while directly improving signal quality and effectively reducing errors in physiological data acquisition. The spacing between the LEDs 60a and the photodiodes 60b is optimized to reduce backscatter from “blood-less” tissues and the barriers 58 reduce LED 60a to photodiode 60b crosstalk.

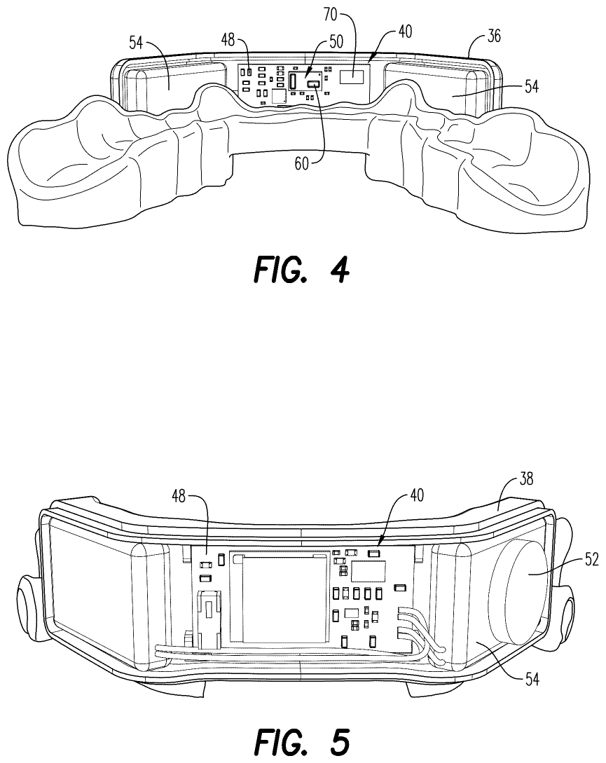

In aspects, the oral appliance 10 may further include one or more of the following additional components: a pressure sensor (not labeled), an airflow sensor (not labeled), a noise detector (not labeled), an actigraphy sensor (not labeled), a stimulator (not labeled), and an electroencephalogram (EEG) sensor 70 (FIG. 4) configured to differentiate various EEG rhythms and to determine, based on these detected rhythms, the various consciousness and sleep states of the user. It is contemplated that the oral appliance 10 may be able to assist with the diagnosis of brain related health conditions, such as epilepsy, by way of recording the user's brain activity—such brain activity may be collected by a data recorder (not labeled) for review and analysis. For further details regarding the EEG sensor 70, reference may be made to U.S. patent application Ser. No. 16/781,417 filed Feb. 4, 2020 (now U.S. Pat. No. 11,375,951), the entire contents of which are incorporated by reference herein. The EEG sensor 70 may be positioned at a portion of the mouthpiece 20 that is adjacent a buccal side of a maxillary bone of the user when the oral appliance is worn. That is, the EEG sensor may be located over the alveolar mucosa between the upper gums and inner lip/cheek of the user.

According to an aspect, the oral appliance 10 may include a transceiver (not labeled). The transceiver may be configured to remotely monitor any additional components provided on and/or within the mouthpiece 20. In an embodiment, the transceiver may be configured for use with a customized web-based application for a handheld wireless communication device. The customized web-based application may include features such as, a graph of the user's sleep position and chart and/or graphical data related to oxygen saturations of hemoglobin and the pressure applied to occlusal surfaces of the user's dentition. According to an aspect, the customized web-based application may include data related to the user's heart rate. In an embodiment, the transceiver communicates with handheld wireless communication devices having Bluetooth® capabilities. The transceiver may be communicable with handheld wireless communication devices, such as, for example, computers, smart watches, smart phones, and the like.

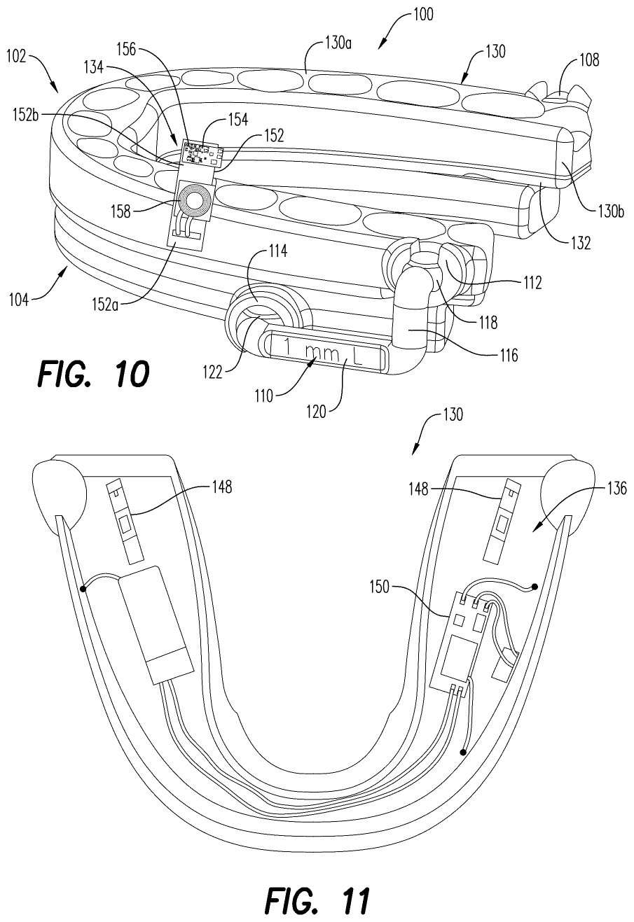

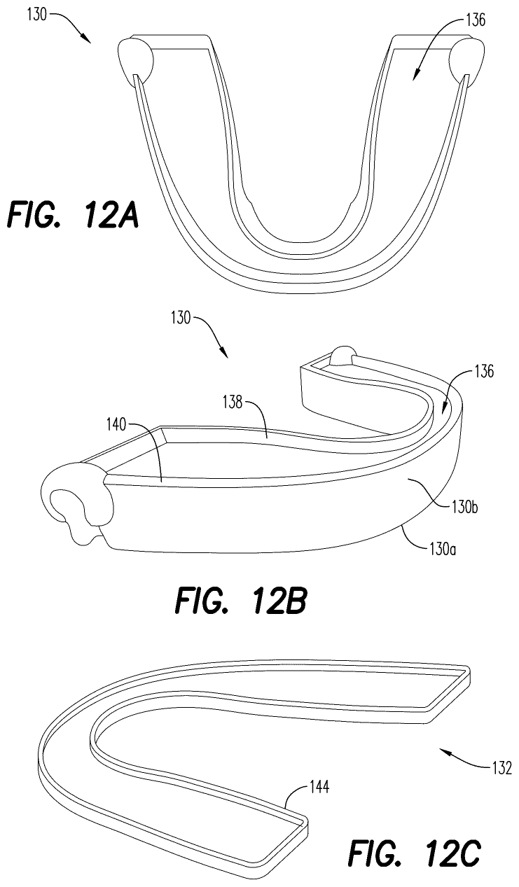

In an embodiment, and with particular reference to FIGS. 10-14, an oral appliance 100 for treatment of sleep apnea in a user is provided. The oral appliance 100 generally includes an upper mouthpiece 102 for positioning over upper dentition of a user, a lower mouthpiece 104 for positioning over lower dentition of the user, an electronic assembly 134 coupled to the upper mouthpiece 102, and a pair of bilateral struts 108, 110 for coupling the upper and lower mouthpieces 102, 104 together. Each of the upper mouthpiece 102 and the lower mouthpiece 104 includes a socket 112, 114 coupled to opposing outer walls thereof. In aspects, the sockets 112, 114 may be monolithically formed with the upper mouthpiece 102 and the lower mouthpiece 104.

In some embodiments, an IMU may be coupled to the lower mouthpiece 104. In some such embodiments, the IMU may be coupled to an anterior portion of the lower mouthpiece 104. More generally, various embodiments described hereby may include an oral appliance with one or more sensors (e.g., sound measuring device, IMU, EMG, etc.). Further, the one or more sensors may be selectively positioned in an upper and/or lower portion of the oral appliance. Additionally, the sensors may be wired and/or wireless. For example, wireless signals may be utilized for communication between an upper portion (e.g., upper mouthpiece) of an oral appliance and a lower portion (e.g., lower mouthpiece) of an oral appliance.

Each of the struts 108, 110 includes an elongate first portion 116 having a first projection 118 configured for receipt in the socket 112 of the upper mouthpiece 102, and an elongate second portion 120 extending perpendicularly from the elongate first portion 116 and having a second projection 122 configured for receipt in the socket 114 of the lower mouthpiece 104s. The oral appliance 10, in combination with the struts 108, 110, may be configured to position the bottom jaw of a user in a direction that is forward from the natural position of the user's jaw to aid in the treatment of sleep apnea in the user. The best jaw position for the user may be determined by a dentist and prescribed to a lab that is tasked with fabricating the oral appliance 10. Further details regarding the sockets 112, 114 and the struts 108, 110 may be found in U.S. patent application Ser. No. 17/737,470 filed May 5, 2022, the entire contents of which being incorporated by reference above.

The upper mouthpiece 102 includes a housing 130, a cover 132, and an electronic assembly 134 coupled to the housing 130. The housing 130 has a first portion or occlusal portion 130a (e.g., a top portion) configured to engage an occlusal surface of a user's dentition, and an opposite second portion 130b (e.g., a bottom portion) defining an inner chamber 136. The inner chamber 136 may extend along the entire arcuate length of the upper mouthpiece 102. The second portion 130b of the housing 130 has an outer peripheral edge 138 defining a groove 140, and the cover 132 has an outer peripheral edge or ridge 144 configured for complimentary engagement with the groove 140. The cover 132 of the housing 130 is secured to the second portion 130b of the housing 130 to seal the inner chamber 136 from an external environment. It is contemplated that a sealing mechanism may be provided between at least a portion of the outer peripheral edge or ridge 144 and the groove 140. A biocompatible adhesive may be provided between the groove 140 and ridge 144 to secure the cover 132 and the housing 130 to one another. It is contemplated that the biocompatible adhesive may have sealing capabilities.

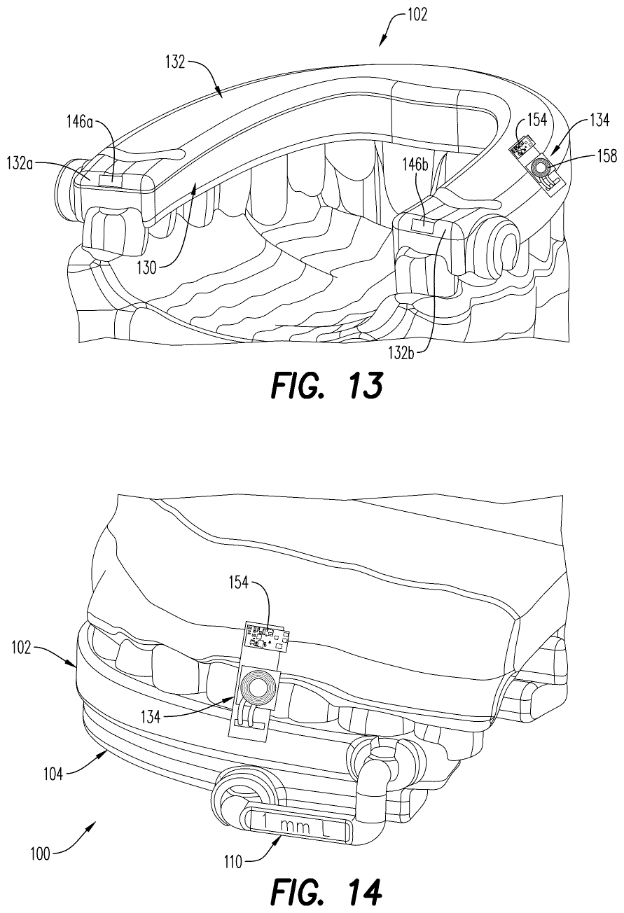

The cover 132 has a pair of posterior ends 132a, 132b (FIG. 13) each defining a port 146a, 146b therethrough in communication with the inner chamber 136. In aspects, the second portion 130b of the housing 130 may define ports in posterior ends thereof. The ports 146a, 146b may each have a filter or impermeable mesh cover to prevent fluids or debris from entering the inner chamber 136 while permitting sound to pass therethrough. The upper mouthpiece 102 may include a pair of microphones 148, such as, for example, Micro-Electro-Mechanical Systems (“MEMS”) microphones (e.g., the INFINEON MEMS mic), positioned in the inner chamber 136 adjacent the respective ports 146a, 146b. The microphones 148 are configured to detect at least one of the user's normal breathing, abnormal breathing, changes in the user's breath sound intensity, or the user's snore sound quality. The microphones 148 may be configured to detect upper airway collapse in instances of obstructive sleep apnea. It is contemplated that the microphones 148 may be configured to identify specific breathing patterns associated with sleep-disordered breathing. A printed circuit board 150 may be provided in the inner chamber 136 in addition to other electronic components, including one or more of a pressure sensor, an airflow sensor, a noise detector, an actigraphy sensor, a stimulator, a peripheral oxygen saturation (SpO2) sensor, a combined gyroscope and accelerometer, or an electroencephalogram (EEG) sensor.

The microphones 148 may be pre-amplified and configured to record or sample sound at a frequency of about 10 kHz (thereby digitizing the analog sound signal captured). More generally, a sound measuring device may record or sample sound at a frequency between 1 and 20 kilohertz (kHz), such as 2, 4, 6, 8, 10, 12, 14, or 16 kHz. The frequency may be selected to obtain acceptable data fidelity while maximizing battery life (e.g., the lower the sampling rate the longer the battery life). In some embodiments, the frequency may be selected by a user. More generally, the sampling rate of one or more of the sensors disclosed hereby may be adjusted or set to maintain acceptable data fidelity while maximizing battery life.

According to an aspect, the sound measuring device (e.g., microphones 148) is configured as a sensor that is configured to measure unique patterns of breath sounds. The unique patterns of breath sounds including snore sound waves and/or air exchange intensity, as detected with a MEMS mic, can represent unique phenotypical characteristic associated with OSA. Through predictive modeling algorithm, we are able to use the collected data at a sampling rate of 8 kilohertz per second and determine the site of collapse, the onset of obstruction, severity of OSA, and/or trigger to initiate therapeutic modality such as tongue stimulation. The compilation of data collected over time, will increase the sensitivity of the predictive value.

According to an aspect, the microphones 148 are configured to filter signals with:
high pass τ=0.004 s [order 2]; and
low pass τ==0.0005 s [order 2],
wherein a root-mean-squared (RMS) signal (moving median ⅙th) is calculated to capture the magnitude of the sound. RMS computes the square root of the mean square of the instantaneous values of the signal. This processing, when applied to each sample of a continuous signal, simply makes all negative values of a signal positive, while keeping positive values as positive. Subsequently, a moving time average may be performed to describe the changes in the RMS signal magnitude over time (i.e. within a breath). Specifically, a moving median may be used, which may describe the median value over a 0.33-second window (window half-width w=0.167 seconds; the output signal at time t is the median value of the input data points from time t−w to t+w seconds).

In at least one configuration, the microphones 148 include a S-VM3000-c Vesper-Mouser.

According to an aspect, the upper mouthpiece 102 includes a processor operably coupled to the microphones 148. The processor in combination with the microphones 148 are configured to identify the user's total sleep time, and total number of sleep disturbances followed by arousals during respiratory events. The microphones 148 are configured to detect changes in the user's air exchange intensity, and the processor is configured to translate the detected changes and trigger a stimulator to stimulate a muscle in the user's oral cavity to reverse the obstruction of the upper airway.

The microphones 148 in combination with the processor are configured to detect at least one of snoring and awake periods or arousal during the user's sleep.

According to an aspect, the upper mouthpiece 102 further includes a storage chip configured to store and record data; and a battery configured to provide power to at least one of the sound measuring device, the processor, and the storage chip. According to an aspect, the data can also be lived streamed and stored externally, if desired by the operator.

As shown in FIGS. 10, 13, and 14, the electronic assembly 134 may be external to the upper mouthpiece 102 and may extend upwardly from the upper mouthpiece 102. In aspects, the electronic assembly 134 may be encased in a transparent outer housing (not explicitly shown). The electronic assembly 134 includes a substrate 152, an oxygen sensor 154, a printed circuit board 156, an optical housing (not labeled) supported on the printed circuit board 156, and a charging coil 158. The substrate 152 has a first end portion 152a coupled to the housing 130, and a second end portion 152b extending away from the housing 130 in a direction toward gingiva of a user. The first end portion 152a of the substrate 152 is coupled to the housing 130 of the upper mouthpiece 102 and the second end portion 152b of the substrate 152 supports the printed circuit board 156 thereon. The optical housing is supported on the printed circuit board 156 and the oxygen sensor 154 is supported in the optical housing and electrically coupled to the printed circuit board 156. The optical housing may be substantially similar to or identical to the optical housing 56 (FIG. 7) described above.

The oxygen sensor 154 is configured to be oriented toward alveolar mucosa or gingiva of the user to determine an oxygen level of the user. The oxygen sensor 154 may be a reflectance pulse oximeter configured to monitor/sense the oxygen saturation of a user by analyzing the change in color of the user's blood. The reflectance pulse oximeter 154 may measure the pulse rate of the user, typically in beats per minute, based on variations and/or deviations in the user's oxygen saturation. An exemplary pulse oximeter may use light-based technology to sense the actual oxygen saturations of hemoglobin of the user. According to an aspect, the pulse oximeter 154 may be substantially similar to or identical to the pulse oximeter 60 described above.

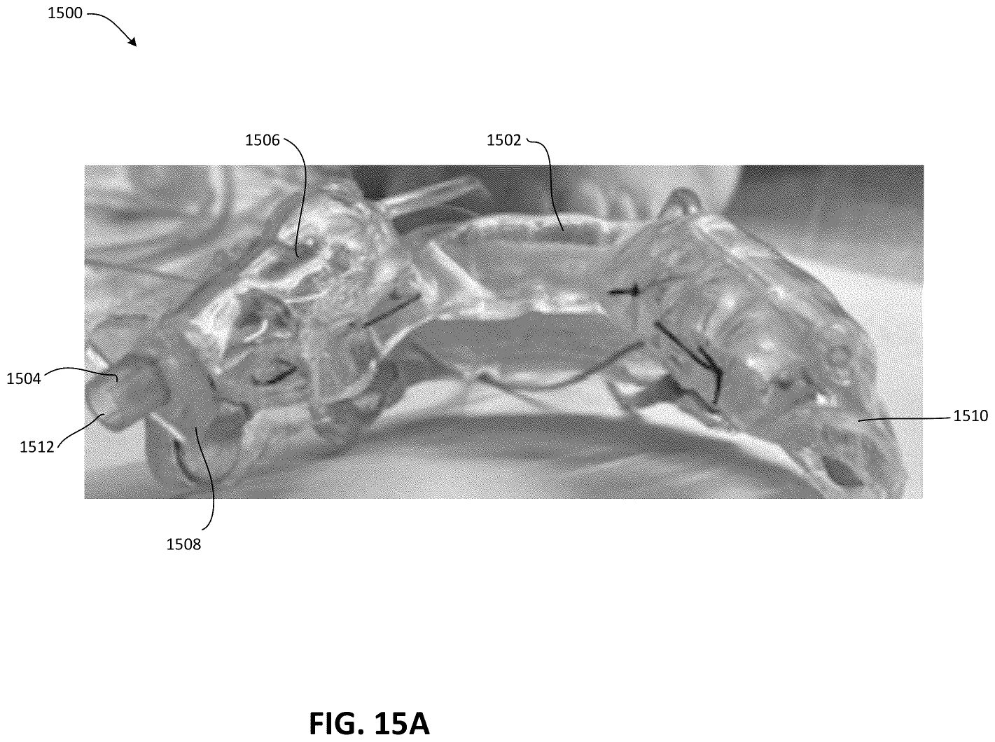

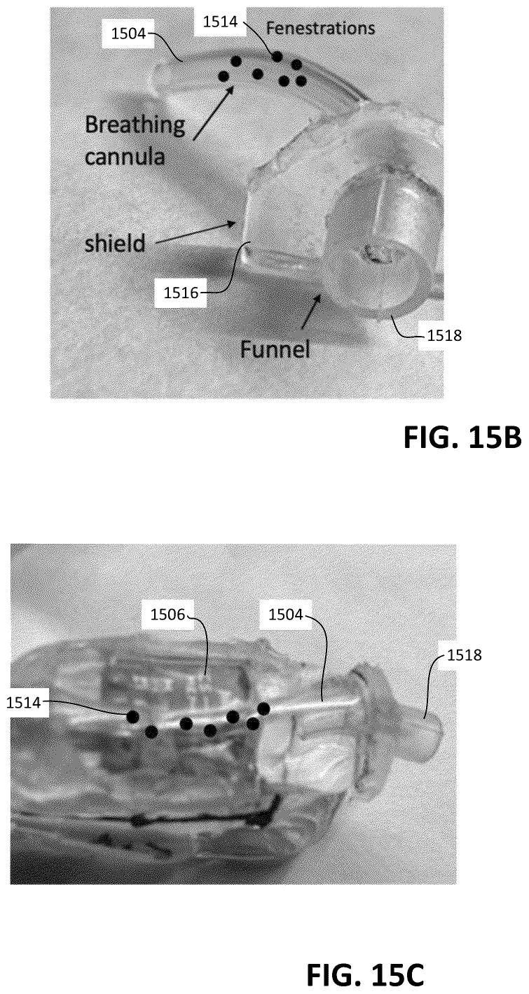

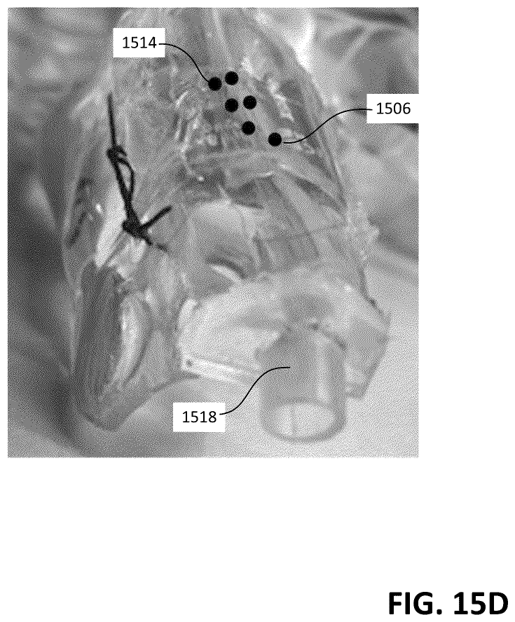

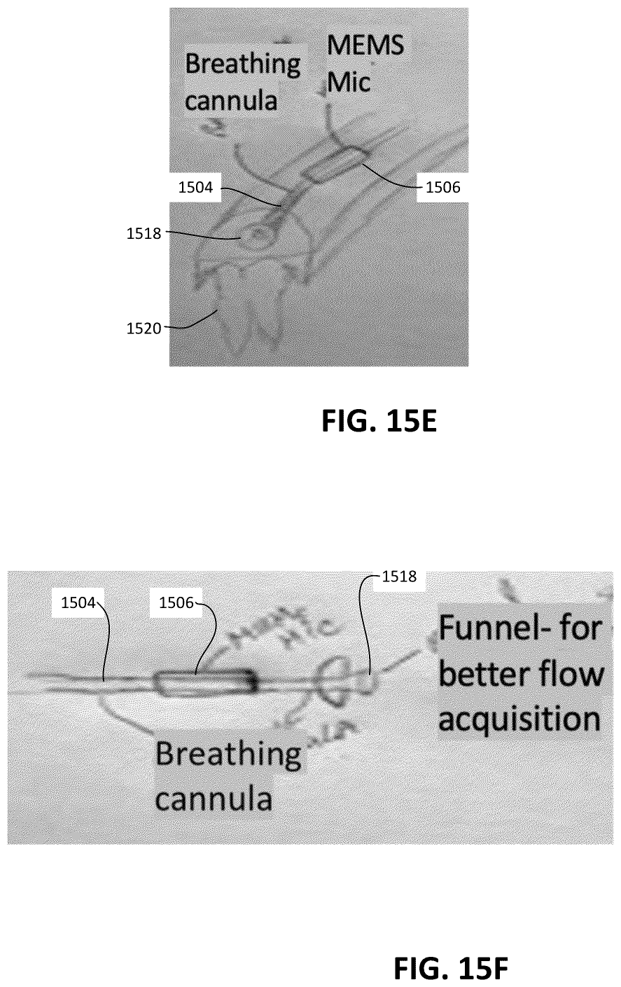

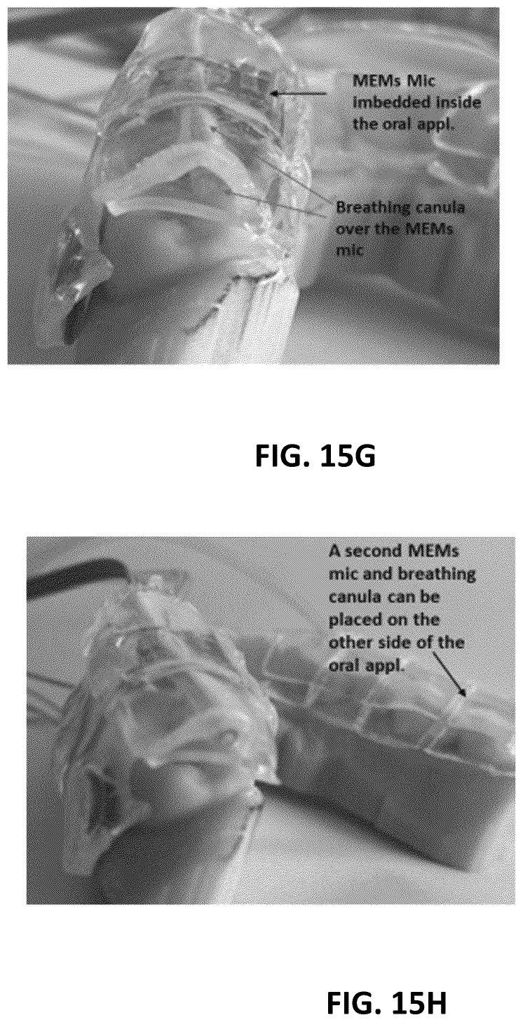

FIG. 15A is a perspective view of an oral noise monitor 1500, according to an aspect. The oral noise monitor 1500 includes a mouthpiece 1502 having a first end 1508 and a second end 1510. According to an aspect, the first end 1508 and the second end 1510 are both at the posterior portions of the mouthpiece 1502 in a spaced apart configuration from each other. In an aspect, the oral noise monitor 1500 may be combined or integrated with an oral appliance (e.g., oral appliance 102). In many embodiments, the oral noise monitor 1500 may be combined with an IMU in an oral appliance.

A cannula 1504 may extend through the mouthpiece 1502. According to an aspect, the cannula 1504 extends from the first end 1508 to the second end 1510 of the mouthpiece 1502. The cannula 1504 has a first opening 1512 and a second opening (not shown), each opening being configured to receive airflow from the user having the mouthpiece 1502 positioned in their oral cavity.

According to an aspect, a housing/compartment (not shown) may be provided for housing a noise sensor 1506 in such a configuration that the noise sensor 1506 is hermetically sealed. The cannula may extend through the housing. In an aspect, a funnel 1518 (FIG. 15B) has a larger inner diameter than the cannula, which may reduce the likelihood of fluid passing through the funnel and into the cannula due at least in part to the change in hydrostatic pressure between an interior of the funnel and an adjacent interior of the cannula.

FIG. 15B is a perspective view of a portion of the cannula 1504 for use with the oral noise monitor 1500. As illustrated, at least one of the first end 1508 and the second end 1510 of the mouthpiece 1502 includes the funnel 1518. A first funnel may be connected to the first end portion (substantially adjacent to the first opening of the cannula) and a second funnel may be connected to the send end portion (substantially adjacent to the second opening of the cannula). It is contemplated that the funnel 1518 may capture more air and then the captured air will be compressed or confined in the cannula 1504 and brought towards the location of the noise sensor 1506 so that a more accurate reading can be obtained.

It is contemplated that a shield 1516 may be circumferentially disposed around the funnel 1518. According to an aspect, the shield 1516 may be secured to both the funnel 1518 and the cannula 1504. The shield 1516 may help to prevent any moisture or fluids from going into the area where the noise sensor 1506 is positioned.

One or more fenestrations 1514 may be formed through the cannula 1504. The fenestrations 1514 may help to allow the user's airflow to exit the cannula 1504 and go towards the noise sensor 1506. According to an aspect, the fenestrations 1514 are formed so that they are within the housing that houses the noise sensor 1506.

FIG. 15C illustrates a plurality of fenestrations 1514 formed along a portion of the cannula 1504. The fenestrations 1514 are positioned adjacent the noise sensor 1506.

FIG. 15D also illustrates a plurality of fenestrations 1514 formed along a portion of the cannula 1504. The fenestrations 1514 are positioned adjacent the noise sensor 1506. The fenestrations 1514 shown in FIG. 15C are of a different quantity and pattern than the fenestrations 1514 illustrated in FIG. 15D. It is contemplated that any different number and pattern of fenestrations 1514 may be utilized in the oral noise monitor 1500.

FIG. 15E is a perspective view of an oral noise monitor 1500 including a cannula 1504. The oral noise monitor 1500 is illustrated as being positioned over a user's teeth 1520. In some embodiments, oral noise monitor 1500 may include a lower (or upper) portion of an oral appliance. In some such embodiments, an upper (or lower) portion of the oral appliance may include an IMU. The oral noise monitor 1500 includes a noise sensor 1506, and the cannula 1504 is arranged so that the noise sensor 1506 is adjacent to the cannula 1504. According to an aspect, the noise sensor 1506 includes a MEMS mic.

FIG. 15F is a side view of the oral noise monitor shown in FIG. 15E. The funnel 1518 is shown having an opening that has a wider inner diameter than the inner diameter of the cannula 1504. It is contemplated that use of the funnel 1518 helps to facilitate better air flow acquisition so that it can be tested by the noise sensor 1506 due at least in part to hydrostatic pressure changes as detailed above.

FIG. 15G is a perspective view of an oral appliance including a first MEMs mic embedded in a mouthpiece of an oral noise monitor and a cannula disposed above the MEMs mic, according to an embodiment.

FIG. 15H is a perspective view of the oral noise monitor 1500 of FIG. 15G including a second MEMs mic embedded in the oral appliance. It is contemplated that including more than one MEMs mic may help to obtain greater airflow to be tested by one or more noise sensors 1506.

According to an aspect, the sound measuring devices disclosed hereby may be configured to detect at least one of the user's normal breathing, abnormal breathing, changes in the user's breath sound intensity, and the user's snore sound quality. The sound measuring device may be configured to detect upper airway collapse in instances of obstructive sleep apnea. It is contemplated that the sound measuring device may be configured to identify specific breathing patterns associated with sleep-disordered breathing. A processor or micro-processor may be incorporated within the sound measuring device, such as to detect conditions of a user based on data generated by the sound measuring device. More generally, a processor may be utilized to detect one or more conditions of a user based on data generated by one or more sensors (e.g., sound measuring device and/or IMU). These conditions may include at least one of normal breathing, abnormal breathing, awake periods, breath intensity, changes in breath sound intensity, snore sound quality, upper airway collapse, obstructive sleep apnea, sleep disordered breathing, total sleep time, sleep disturbances followed by arousal, jaw clenching, grinding, swallowing, a respiratory disease, or a neurodegenerative disorder. Respiratory diseases may include upper and/or lower respiratory diseases. The neurodegenerative disorders may include Parkinson's or amyotrophic lateral sclerosis.

It is contemplated that the sound measuring device may be a wired or wireless sound measuring device. If a wired sound measuring device is utilized, the wires may be embedded in or secured to the mouthpiece so that they will not be tangled when positioned in the user's oral cavity or destroyed by the user's saliva or salivary minerals and/or other debris in the oral cavity. According to an aspect, the sound measuring device is secured to or embedded within the occlusal wall, such that the sound measuring device is adjacent to the user's upper second molar region. In some embodiments, the sound measuring device may be located in a lower jaw posterior region.

According to an aspect, the sound measuring device is secured to or embedded within a posterior position of the mouthpiece such that when the mouthpiece is positioned in the user's oral cavity the sound measuring device will be close to the back of the user's throat. The sound measuring device may be disposed in a circular housing having a sealed first side and an open second side. According to an aspect, the sealed first side is secured to the mouthpiece by an adhesive and the open second side is exposed to saliva, sounds in the user's mouth, such as breathing sounds and snoring sounds. In various embodiments, the sound measuring device may be protected by a semi-impermeable biocompatible barrier, such as Gore-Tex®, adhered to the body of the appliance to prevent saliva and oral debris from entering the electronics housing of the oral appliance, yet allowing for the breath sounds and flow to be collected unaffected by the MEMS mic

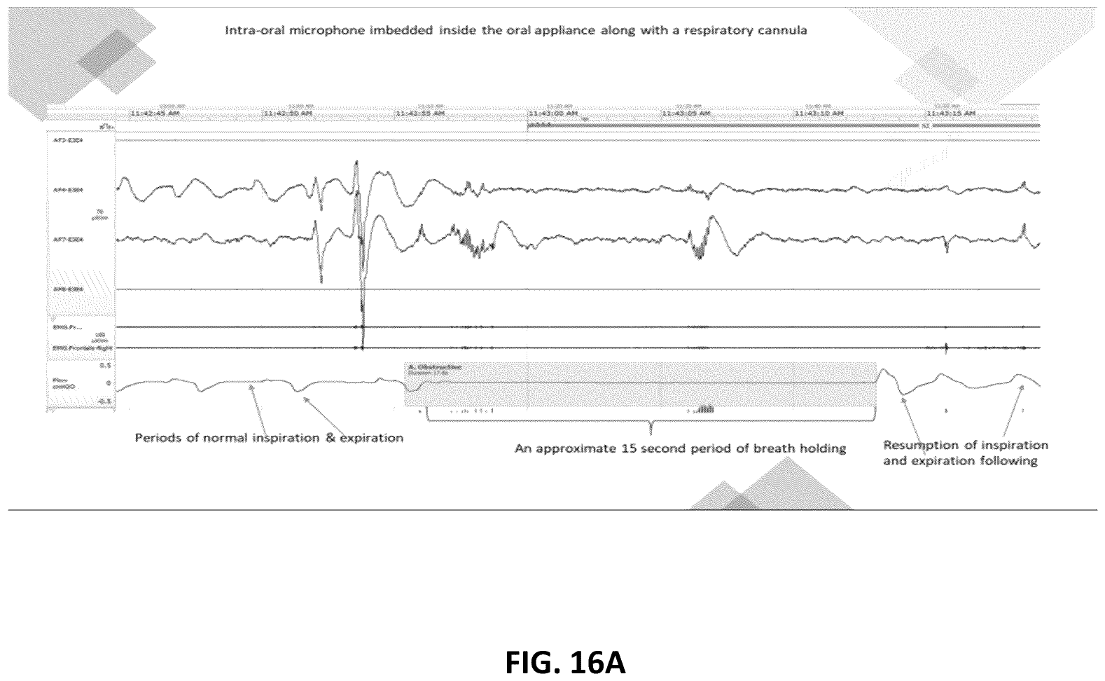

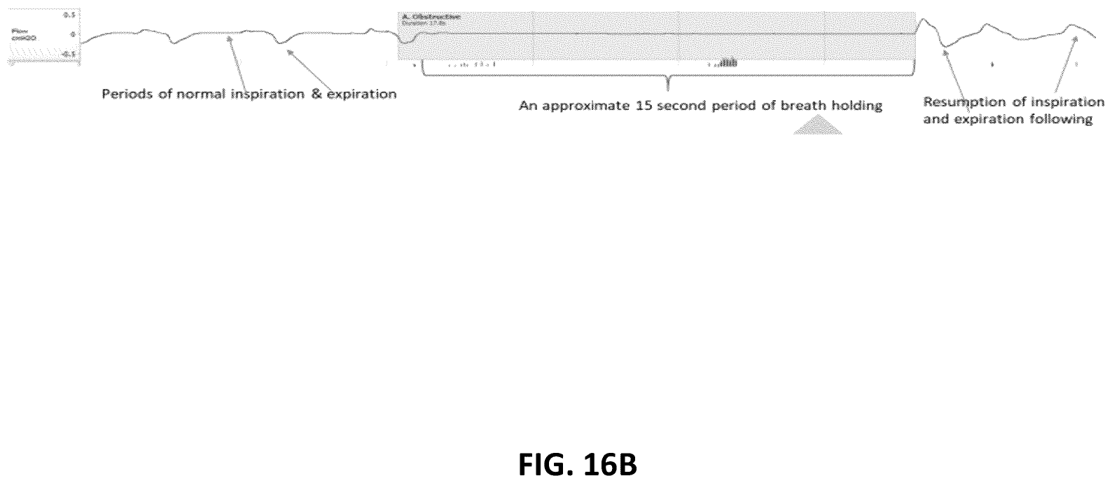

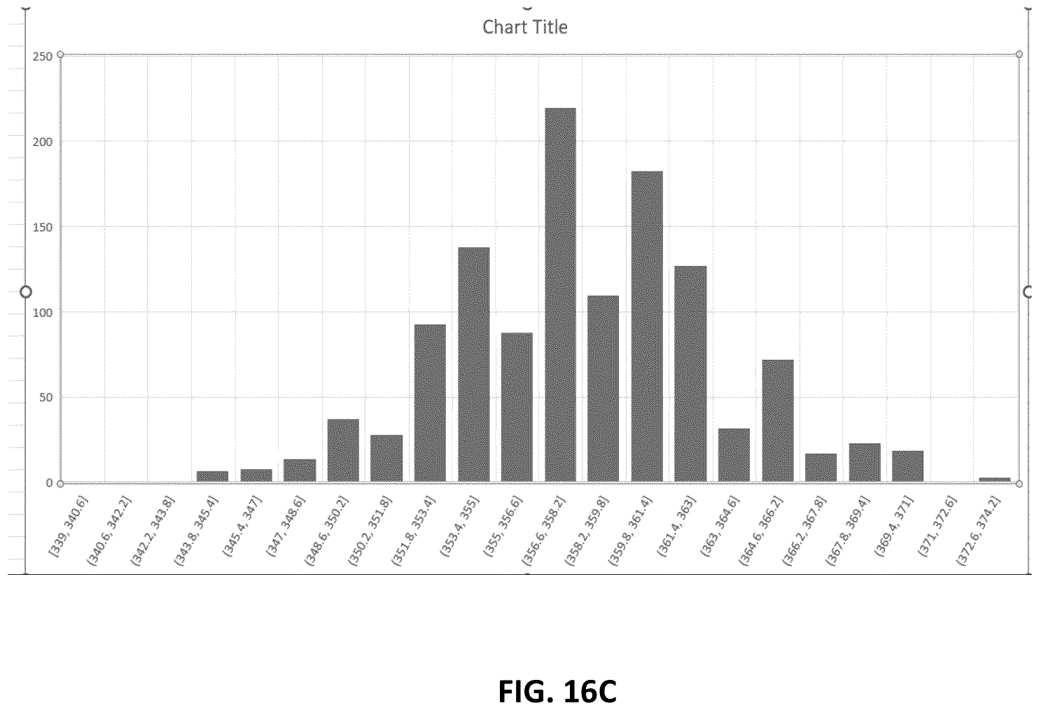

FIGS. 16A-25 illustrate charts that depict the results of sounds that were measuring using a sound measuring device, according to an aspect. The data illustrated in the charts of FIGS. 16A-25 was collected at different time intervals of a snoring test utilizing a sound measuring device (e.g., a microphone) of an oral appliance of the present disclosure. The sound measuring device utilized was a microphone embedded in the oral appliance. The sound measuring device utilized was positioned in a sealed circular housing that was open to one side and coupled to an opposing side by an adhesive. The sound measuring device was positioned at a location inferior to one of the user's upper rear molar.

The objective of the tests was to determine whether loud snoring sounds and quiet breathing sounds, as seen at the trachea, would be clearly detectable in the user's mouth with this oral device. The subject, awake, performed a series of inspiratory snoring sounds followed by periods of normal, quiet breathing. The oral microphone signals were captured well—snoring sounds were detected without clipping and breathing sounds were detected above the noise floor.

FIG. 16A is a chart illustrating data collected by an oral appliance, according to an embodiment.

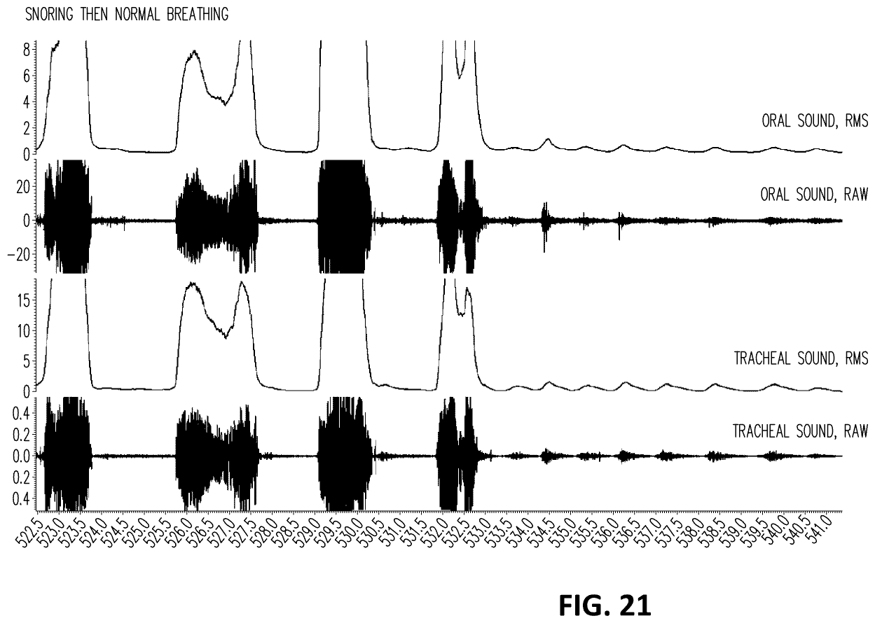

FIG. 16B is a portion of the chart of FIG. 21, illustrating periods of inspiration and expiration, when data is captured using an oral appliance according to any one of FIG. 11 to FIG. 20.

FIG. 16C is a bar chart illustrating normal breathing sounds and snoring sounds captured by an oral noise monitor, according to an embodiment.

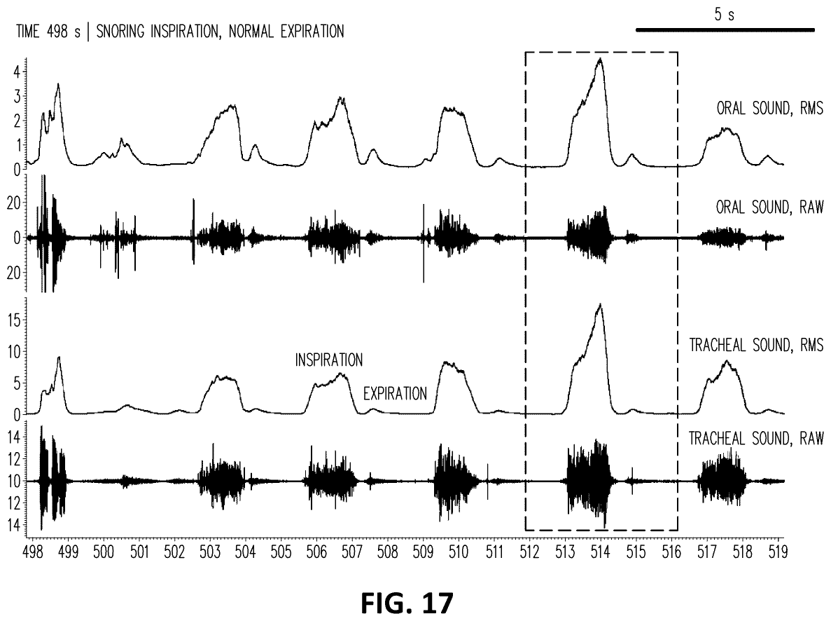

FIG. 17 is a chart that depicts data relating to snoring and normal breathing sounds, collected by a sound measuring device. As illustrated in the chart, sound was measured starting from about 498 seconds into the user's sleep cycle. The user made snoring sounds on inspiration and breathed normally on expiration. The dashed lines around the 512-516 second marks validates the data collected from the sound measuring device given that the data collected by the sound measuring device of the oral appliance is in line with the illustrated standard reference data from collected from an external (e.g., tracheal) microphone.

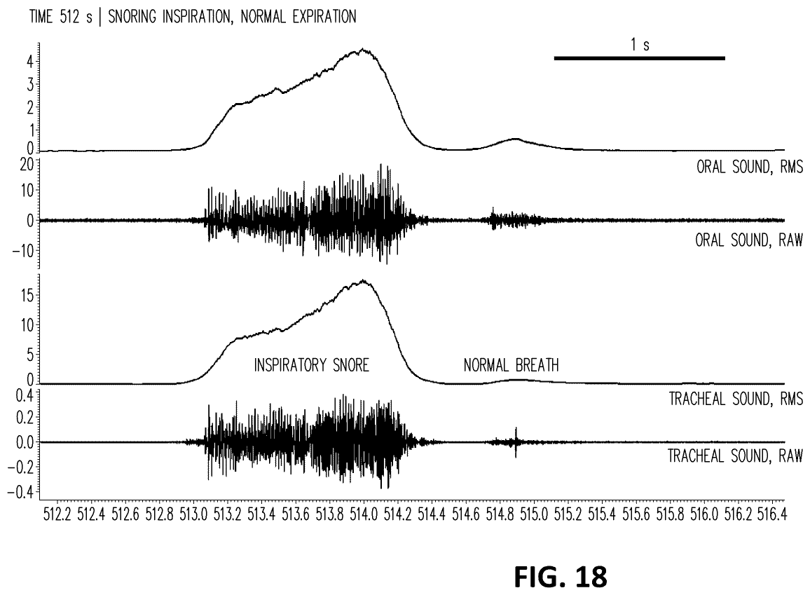

FIG. 18 is a chart that depicts data relating to snoring and normal breathing sounds, collected by a sound measuring device, according to an aspect. As illustrated in the chart, sound was measured from about 512 seconds into the user's sleep cycle. The user made snoring sounds on inspiration and breathed normally on expiration.

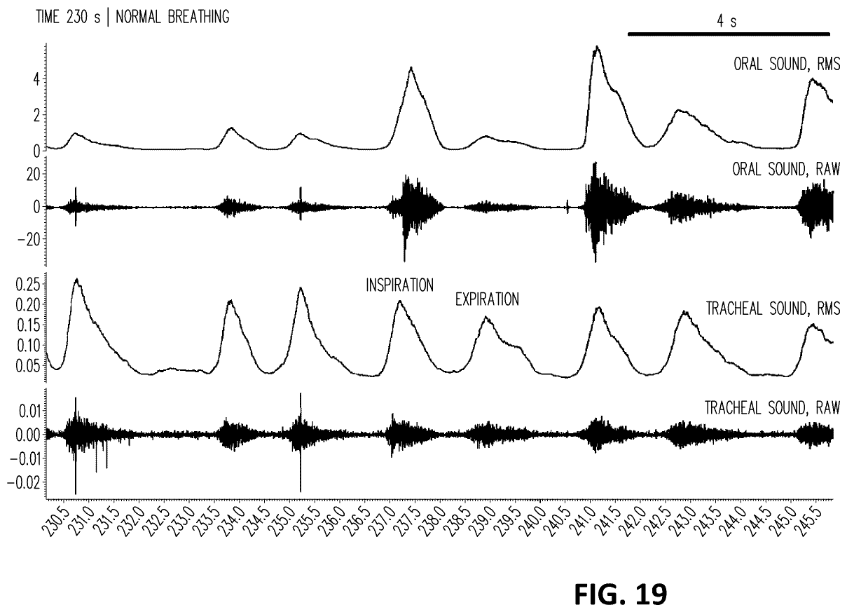

FIG. 19 is a chart that depicts data relating to normal breathing sound, collected by a sound measuring device, according to an aspect. As illustrated in the chart, sound was measured from about 230 seconds into the user's sleep cycle. The user breathed normally during that time period without making any sounds.

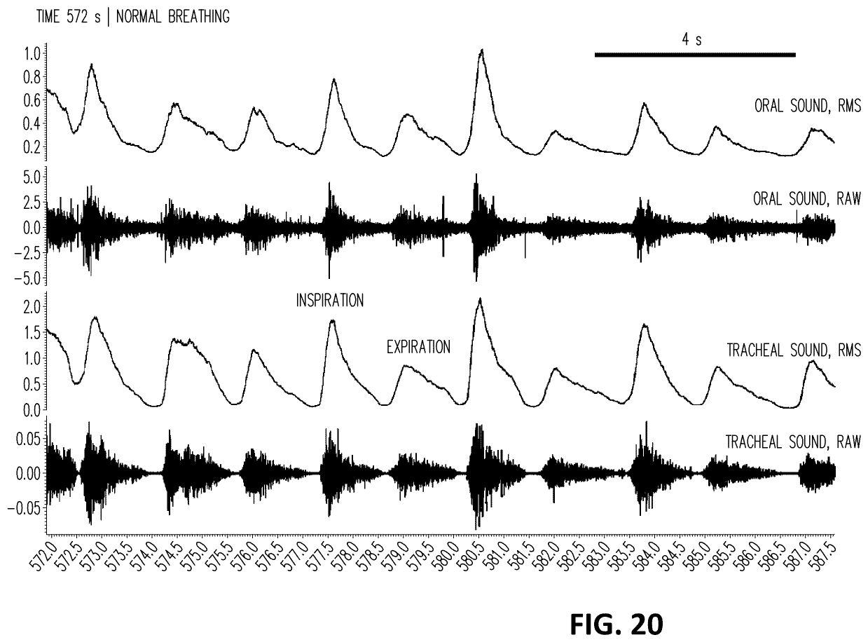

FIG. 20 is a chart that depicts data relating to normal breathing sound, collected by a sound measuring device, according to an aspect. As illustrated in the chart, sound was measured from about 572 seconds into the user's sleep cycle. The user breathed normally during that time period.

FIG. 21 is a chart that depicts data relating to snoring and normal breathing sounds, collected by a sound measuring device, according to an aspect. As illustrated in the chart, the user made snoring sounds then breathed normally.

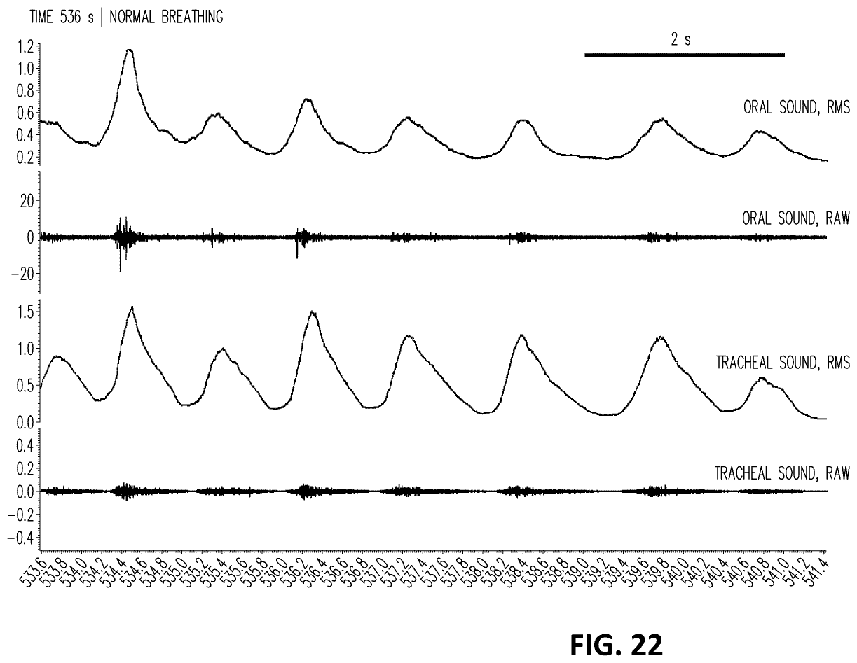

FIG. 22 is a chart that depicts data relating to normal breathing sound, collected by a sound measuring device, according to an aspect. As illustrated in the chart, sound was measured from about 533 seconds into the user's sleep cycle. The user breathed normally during that time period.

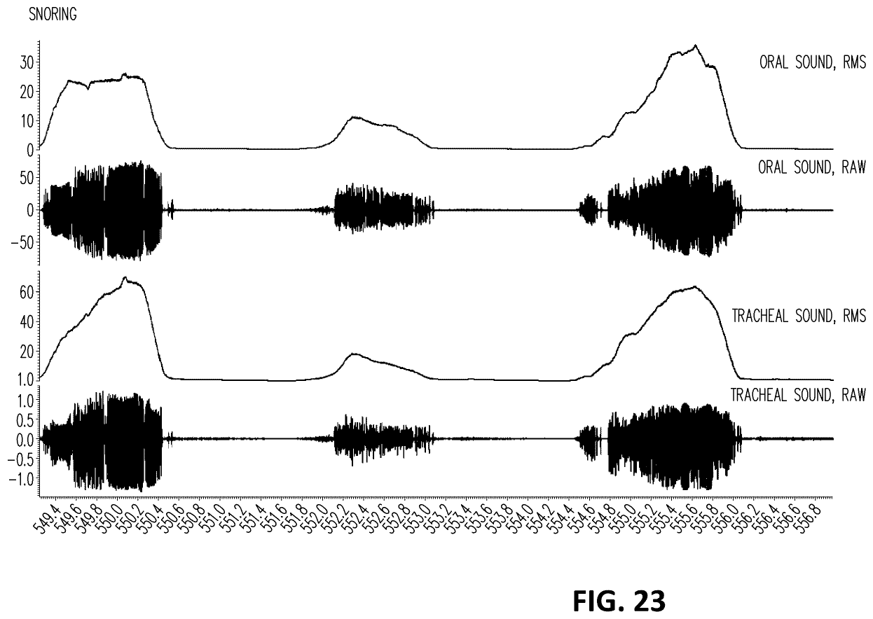

FIG. 23 is a chart that depicts data relating to snoring sounds, collected by a sound measuring device, according to an aspect. The user made snoring sounds during the test.

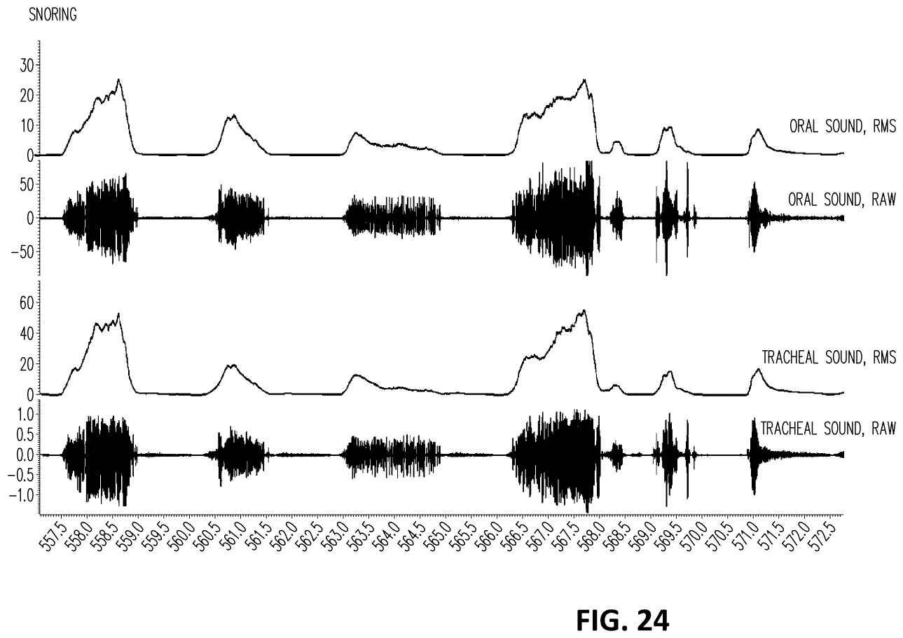

FIG. 24 is a chart that depicts data relating to snoring sounds, collected by a sound measuring device, according to an aspect. As illustrated in the chart, the user made snoring sounds during the test.

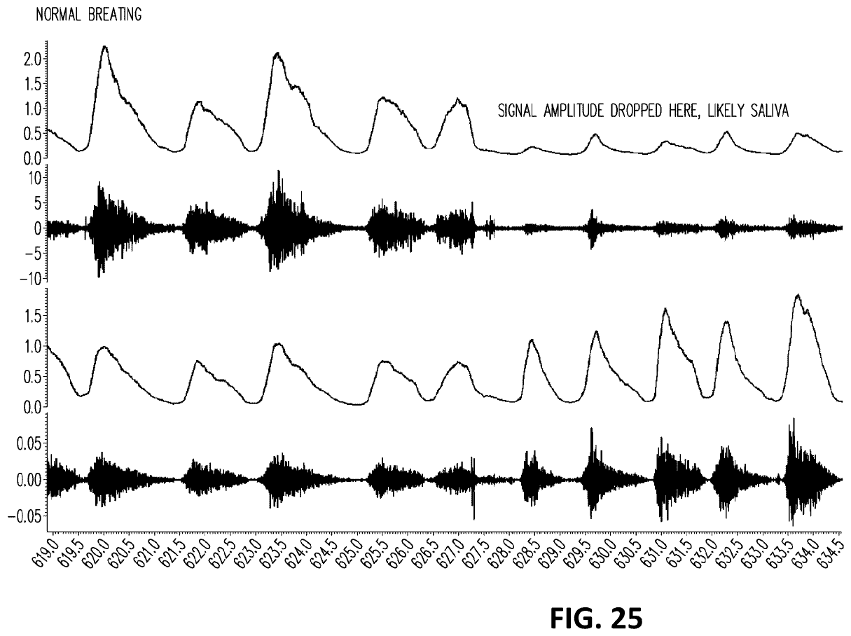

FIG. 25 is a chart that depicts data relating to normal breathing sound, collected by a sound measuring device with a signal amplitude dropped due to saliva, according to an aspect. As illustrated in the chart, the user breathed normally during the test.

In aspects, the present disclosure may provide an oral appliance that includes an inertial measuring unit (IMU) (e.g., the BOSCH 6 axis accelerometer, gyroscope) configured to measure triaxial acceleration and triaxial angular velocity. According to an aspect, the IMU includes a gyroscope and an accelerometer. The gyroscope is configured to output angular velocity signals on three axes in space, while the accelerometer is configured to output linear acceleration signals on three axes in space. According to an aspect, the IMU is a miniature IMU sized so that it can be coupled to the mouthpiece. The miniature IMU may be secured to a buccal, lingual, or occlusal wall of the mouthpiece. The placement of the IMU or miniature IMU may be at the most anterior segment of the user's lower jaw (that is, the location of the user's lower front incisors). It is contemplated that this may an optimal location on the mouthpiece at least because this anatomical location will likely express the most detailed movement during recording. This follows the physics law of “the fulcrum effect”. The closer the point of effort is to the fulcrum (the back of the jaw where the joints and associated muscles and ligaments are), the less effort it will take for the movement at the farthest point to the fulcrum (most anterior part of the jaw)-hence the more sensitive that point is to detecting movement. In some embodiments, the IMU may be configured to detect movement of an upper jaw and/or lower jaw of a user. Further, the IMU may be positioned in an upper and/or lower portion of an oral appliance. It will be appreciated that oral appliances disclosed hereby may include one or more types of sensors. For example, an oral appliance may include a sound measuring device and an IMU. In one such example, the sound measuring device may be included on an upper portion of the oral appliance (e.g., configured to be positioned on an upper jaw) and the IMU may be included on a lower portion of the oral appliance (e.g., configured to be positioned on a lower jaw). In one embodiment, the IMU may be utilized to filter out motion artifacts, such as when utilized in conjunction with an intra-oral pulse oximeter or an intra-oral EEG.

It is contemplated that the oral appliance may be configured for the detection of lower jaw movements. These lower jaw movements may serve as a surrogate bio-signal for the detection of breathing effort, including snoring and arousal during sleep. According to an aspect, the lower jaw movements may be in addition to other bio-signals that are indicative of the presence of sleep apnea in a user. Jaw movement bio-signals can identify specific breathing patterns associated with sleep-disordered breathing, including obstructive sleep apnea (“OSA”) The use of the oral appliance including the IMU or miniature IMU can indicate sleep vs awake stage, and can determine OSA severity levels in those patients. Further analysis using specific algorithms can also identify total sleep time, total number of sleep disturbances followed by arousals during respiratory events or OSA events. According to an aspect, the algorithm may also be used as a predictive model for future systemic issues such as cardiovascular disease (CVD). It is contemplated that the IMU or accelerometer-gyroscope combination may determine jaw clenching, grinding, and/or swallowing which is an indicator for arousals during periods of disturbed sleep. The IMU may also be an indicator of treatment compliance for a patient undergoing treatment for OSA.

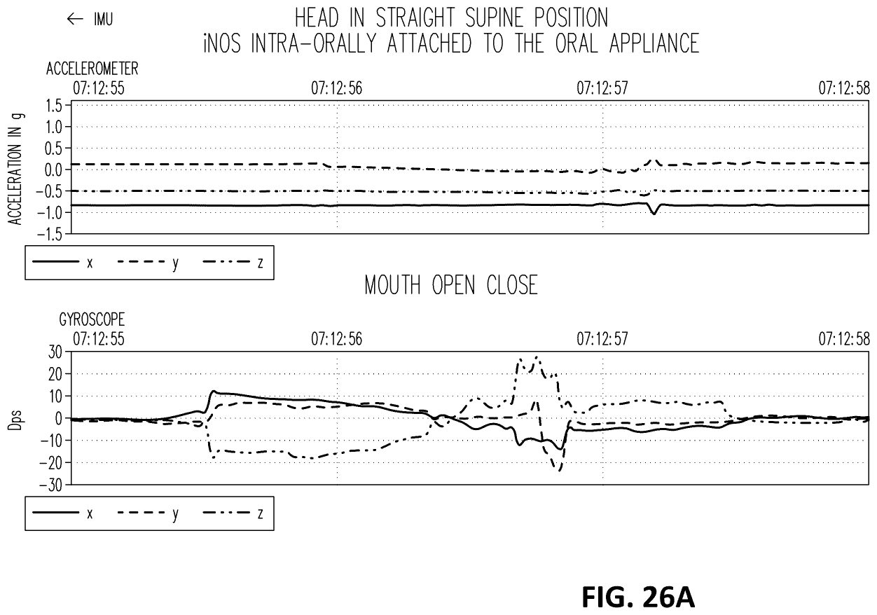

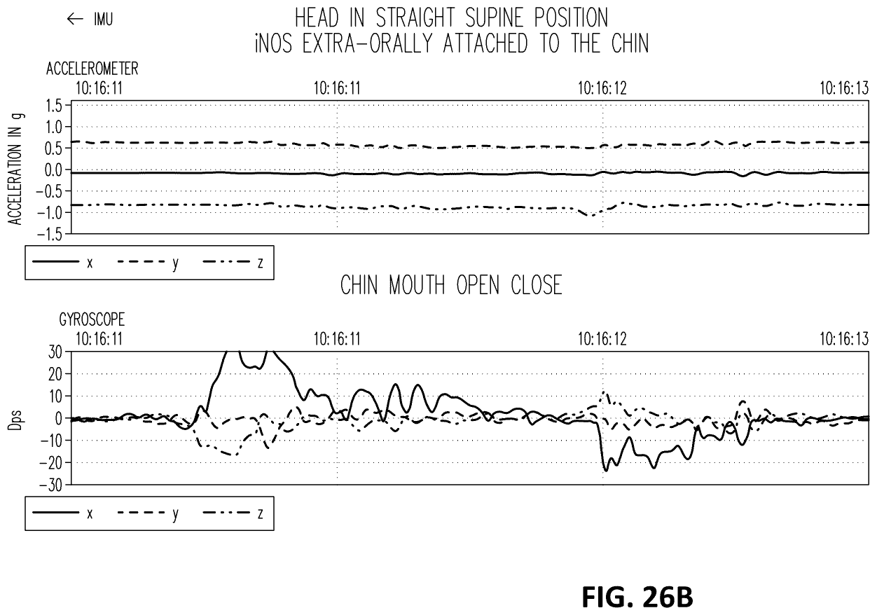

FIGS. 26A-29H include data collected, or generated, by an IMU as described above.

FIGS. 26A and 26B compare data collected by an IMU coupled to an oral appliance of the present disclosure with data collected by an IMU extra-orally coupled to the patient's chin, where a patient is opening and closing their mouth and their head is in a straight supine position. This data may be important in determining whether the patient is a “mouth breather” during sleep and the extent of their mouth breathing.

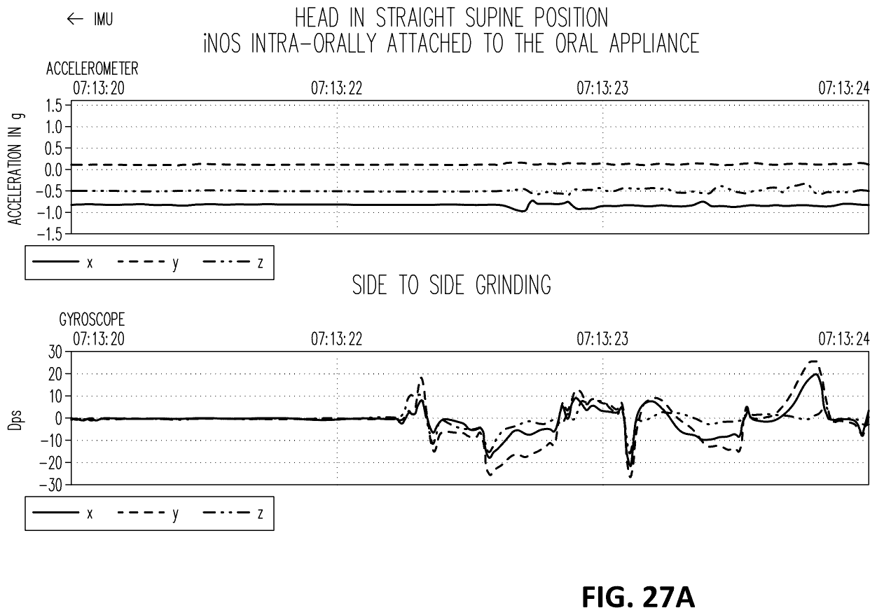

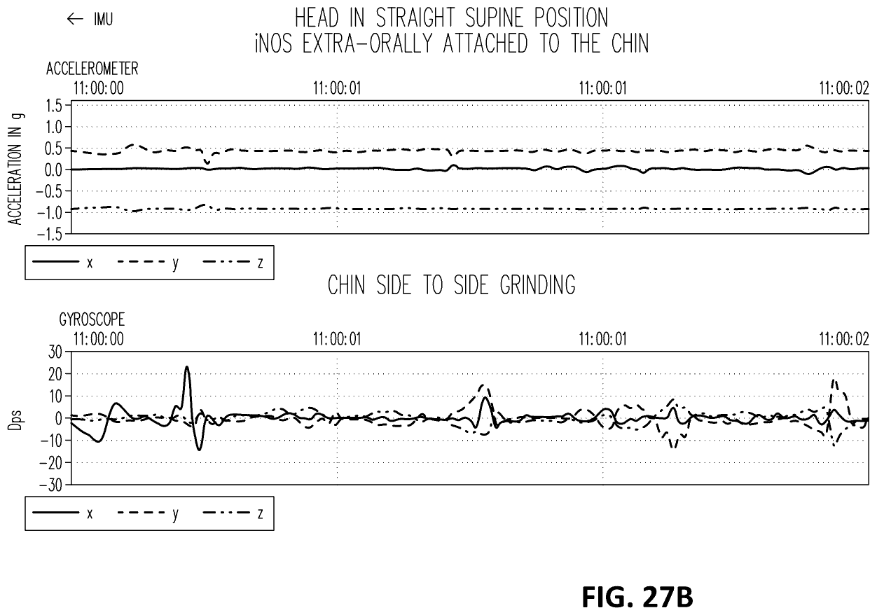

FIGS. 27A and 27B compare data collected by an intra-oral IMU coupled to an oral appliance of the present disclosure with data collected by the IMU extra-orally coupled to the patient's chin, where a patient is grinding their teeth in a side-to-side motion and their head is in a straight supine position. This data may provide an indication of sleep bruxism secondary to obstructive sleep apnea.

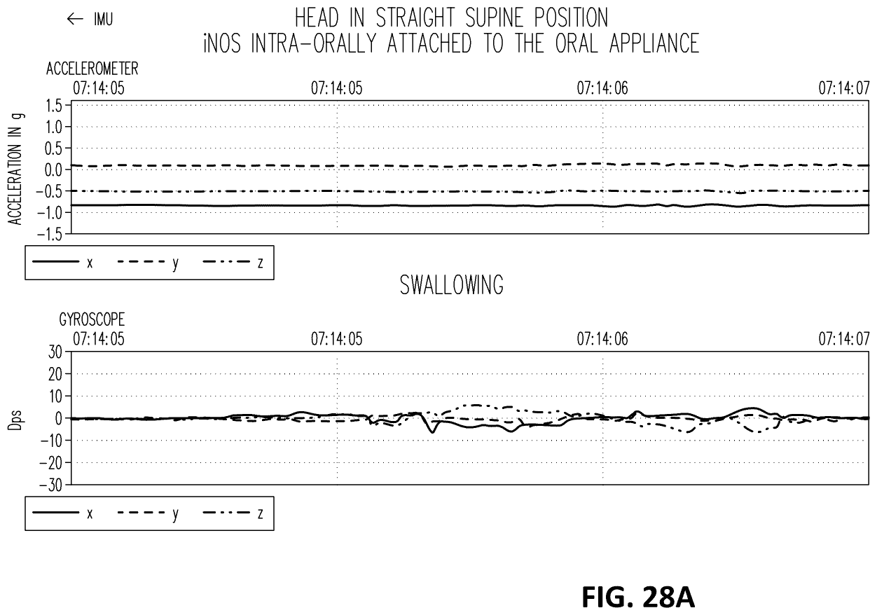

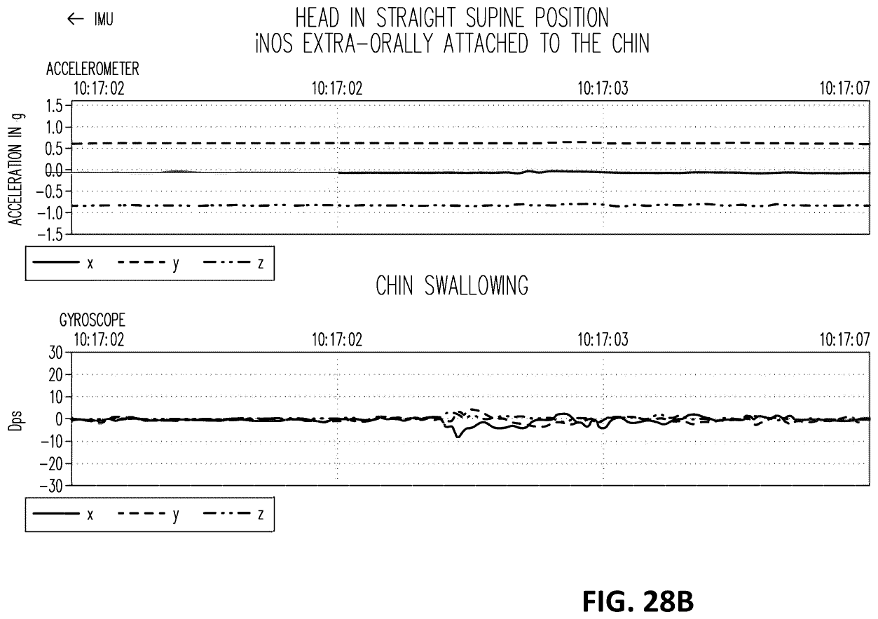

FIGS. 28A and 28B compare data collected by an IMU coupled to an oral appliance of the present disclosure with data collected by the IMU extra-orally coupled to the patient's chin, with a patient swallowing.

Swallowing is a strong indicator of arousal during sleep, most frequently occurring in REM. The intra-oral IMU of the present disclosure has a very distinct and consistent wave shape during swallowing, compared to the external chin sensor.

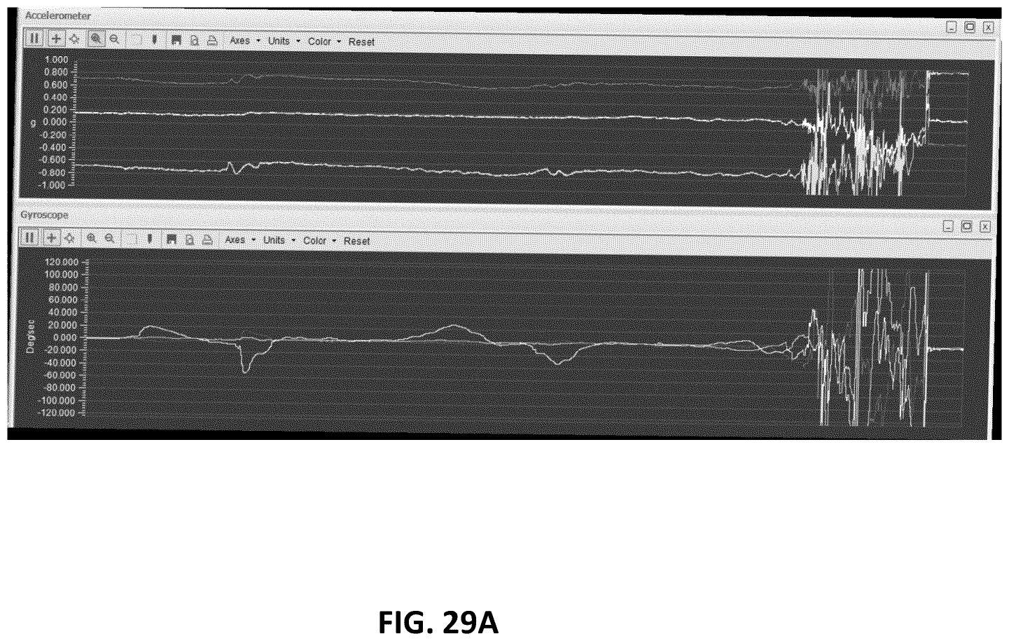

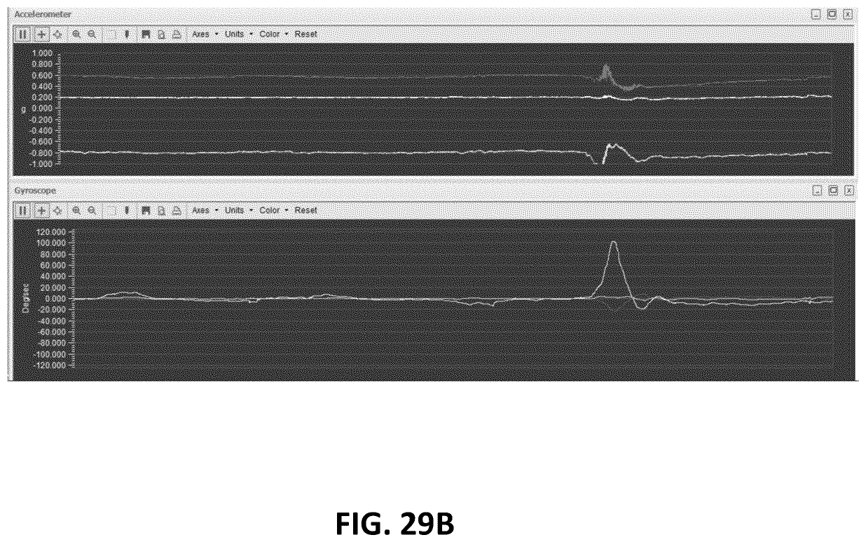

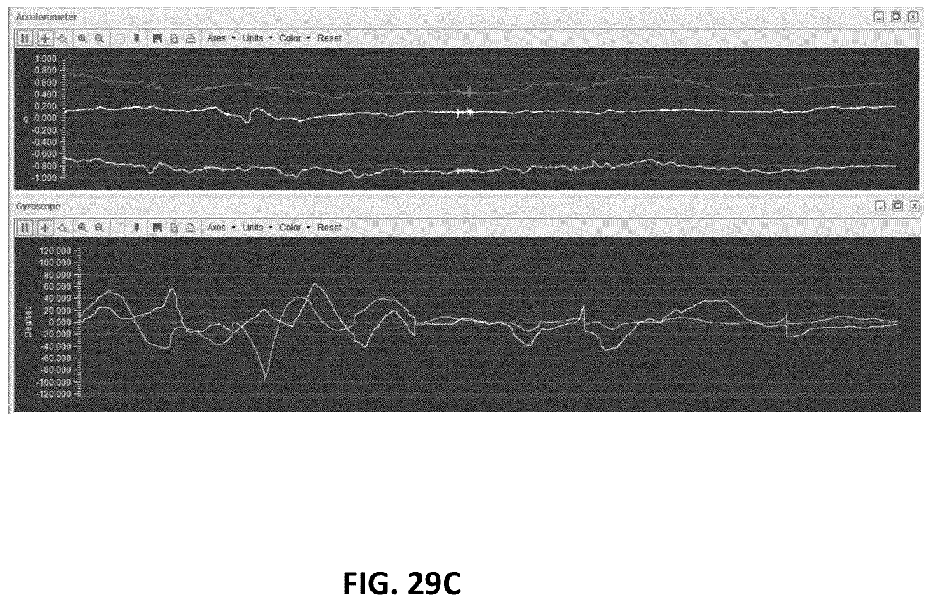

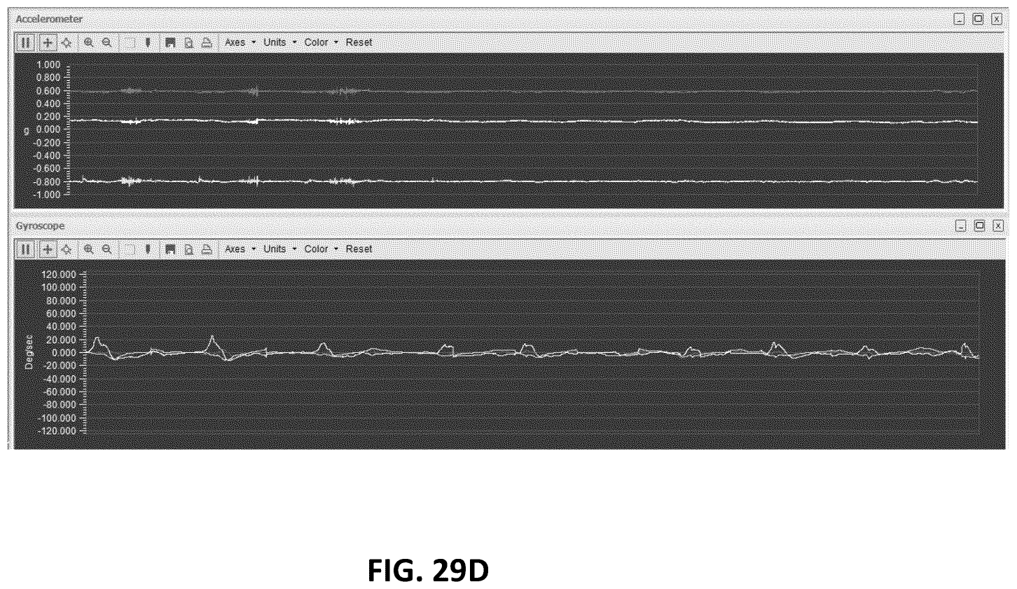

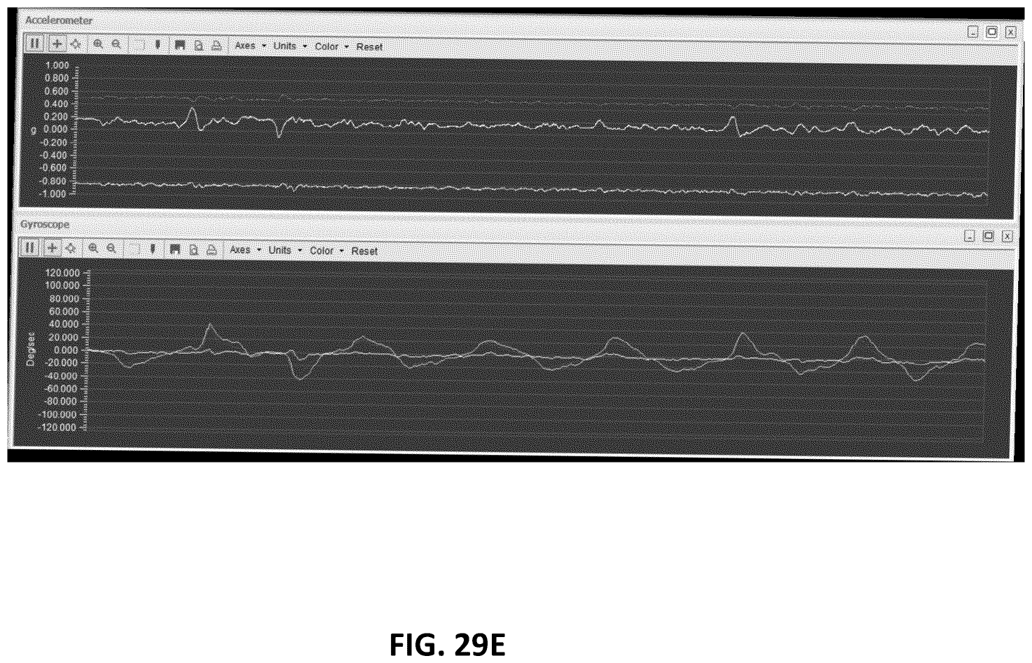

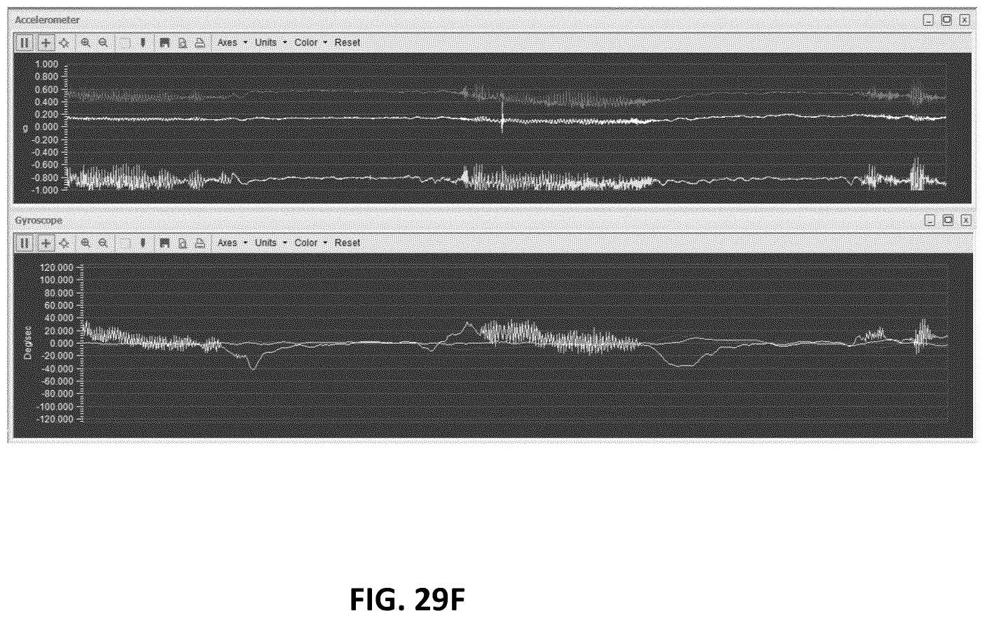

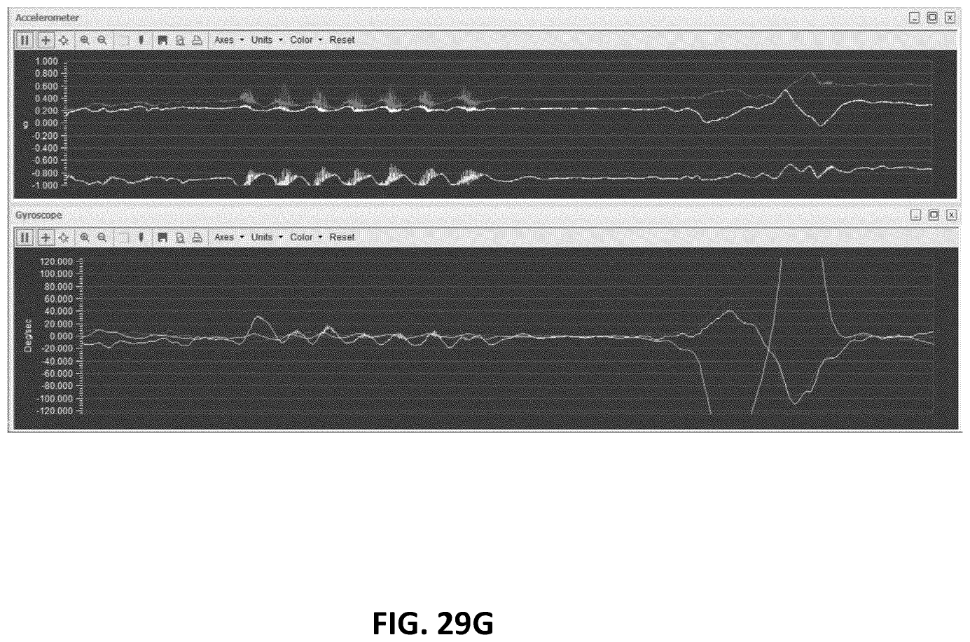

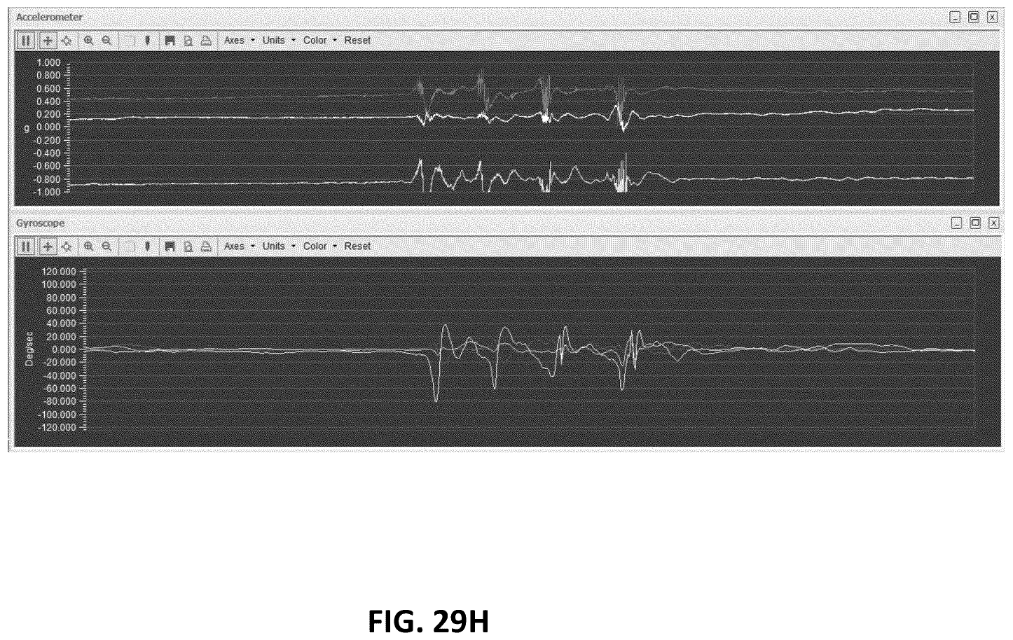

FIGS. 29A-29H are charts illustrating various aspects of data generated by an IMU. More specifically, FIG. 29A is a chart that depicts data relating to breathing movements collected by an inertial measurement unit, according to an aspect; FIG. 29B is a chart that depicts data relating to breathing movements followed by a large recovery breath as collected by an inertial measurement unit, according to an aspect; FIG. 29C is a chart that depicts data relating to large activity levels indicative of wakefulness as collected by an inertial measurement unit, according to an aspect; FIG. 29D is a chart that depicts data relating to jaw clenching movements collected by an inertial measurement unit and indicative of bruxism, according to an aspect; FIG. 29E is a chart that depicts data relating to lateral jaw movement as collected by an inertial measurement unit, according to an aspect; FIG. 29F is a chart that depicts data relating to snoring and movements collected by an inertial measurement unit and indicative of obstructive sleep apnea, according to an aspect; FIG. 29G is a chart that depicts data relating to a wake sound as collected by an inertial measurement unit, according to an aspect; and FIG. 29H is a chart that depicts data relating to a wake sound as collected by an inertial measurement unit, according to an aspect.

Referring to FIG. 29A, data collected (or generated) by the IMU is illustrated. The gyroscope and accelerometer have detected breathing movements, which may serve as a surrogate for a ventilatory drive. In other words, a processor may detect a condition of a user comprising breathing movements of the user based on data generated by the IMU. The breathing movements illustrated are akin to those in quiet breathing, and the period of quiet breathing is terminated by an abrupt increase in activity, a surrogate for arousal from sleep.

Referring to FIG. 29B data collected by the IMU is illustrated. The gyroscope and accelerometer have detected breathing movements that were followed by a large recovery breath. In other words, a processor may detect a condition of a user comprising breathing movements that were followed by a large recovery breath of the user based on data generated by the IMU. The large recovery breath is indicative of a user's arousal from sleep.

Referring to FIG. 29C, data collected by the IMU is illustrated. The gyroscope and accelerometer have detected large activity levels, which are indicative of a user's wakefulness. In other words, a processor may detect a condition of a user comprising wakefulness of the user based on identifying large activity levels in data generated by the IMU.

Referring to FIG. 29D data collected by the IMU is illustrated. The gyroscope and accelerometer have detected jaw clenching movements. These movements may be indicative of sleep bruxism. In other words, a processor may detect a condition of a user comprising bruxism of the user based on identifying jaw clenching movements in data generated by the IMU.

Referring to FIG. 29E, data collected by the IMU is illustrated. The gyroscope and accelerometer have detected lateral jaw movements. In other words, a processor may detect a condition of a user comprising lateral jaw movements of the user based on data generated by the IMU These lateral jaw movements may be detected as cyclic correlated movements in two gyroscope axes.

Referring to FIG. 29F, data collected by the IMU is illustrated. The gyroscope and accelerometer have detected snoring and breathing movements, which are indicative of obstructed breathing or OSA in a user.

Referring to FIG. 29G, data collected by the IMU is illustrated. The gyroscope and accelerometer have detected wake sounds. The wake sounds detected in FIG. 29G is laughter.

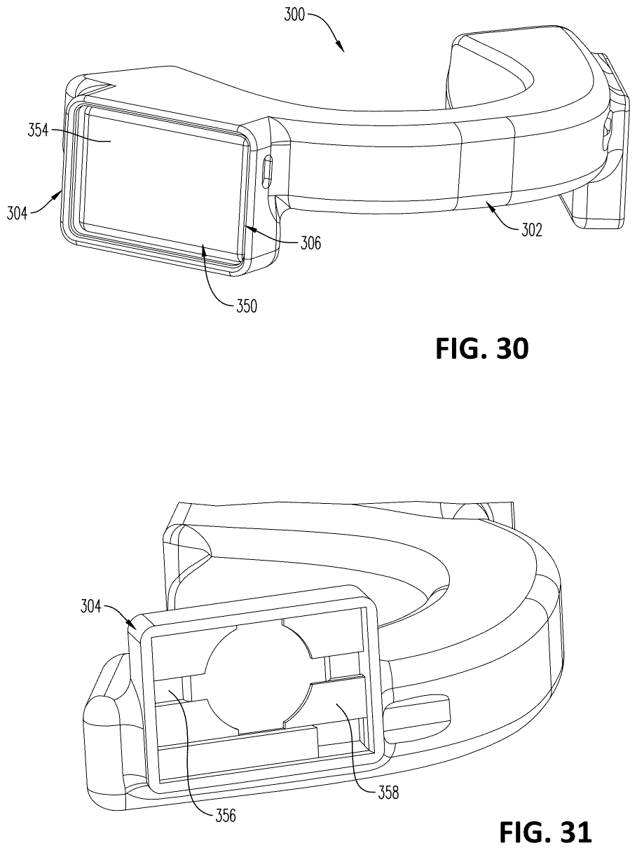

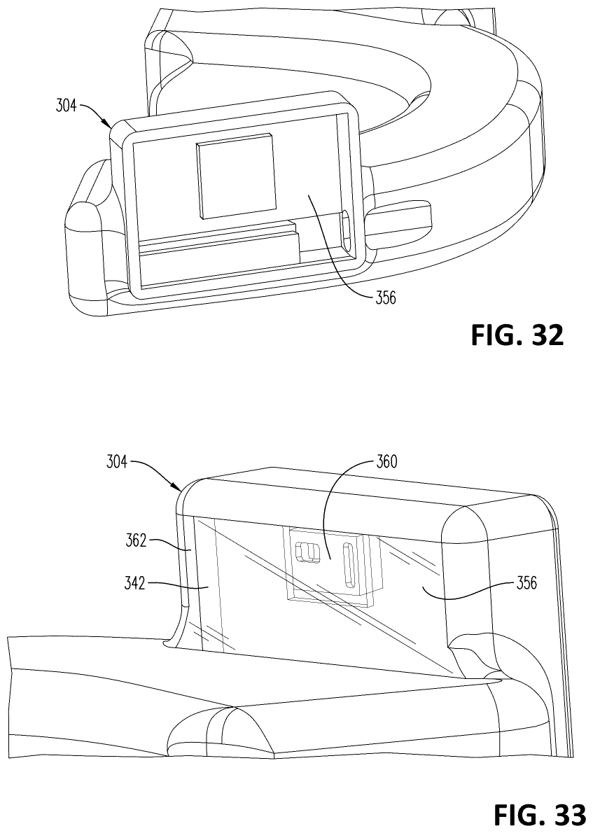

Referring to FIG. 29H data collected by the IMU is illustrated. The gyroscope and accelerometer have detected wake sounds. The wake sounds detected in FIG. 29G is coughing. With reference to FIGS. 30-33, another embodiment of an oral appliance 300 is provided, similar to the oral appliances 10, 100, 200 described above. The oral appliance 300 generally includes a mouthpiece 302 and a sensor assembly 350 positioned in an appendage or box 304 extending perpendicularly away from a posterior end portion of the mouthpiece 302. The sensor assembly 350 may be positioned adjacent an optical window 342 (FIG. 33) of the appendage 304 and includes a charging coil 354, a printed circuit board 356, a spacer 358, and an oxygen sensor 360. The oxygen sensor 360 may be oriented on a sagittal plane of the user's oral cavity and towards buccal gingiva of the user when the oral appliance 300 is worn by the user. The oxygen sensor 360 may be similar to any of the oxygen sensors described above.

The charging coil 354 is stacked over the printed circuit board 356 and separated from the printed circuit board 356 by the spacer 358 to reduce heat that is produced by the charging coil 354 during charging. As such, the spacer 358 keeps excess heat away from the printed circuit board 356. The appendage 304 is specially designed to allow for the charging coil 354 to be housed in a recessed part 306 of the appendage 304, which is about 0.6 mm in thickness relative to the rest of appendage 304. This minimal thickness (e.g., about 0.6 mm) will allow for more efficient contact between the charger (not explicitly shown) and the charging coil 354. The appendage 304 may have another recessed part 362 (FIG. 30) with a minimal thickness relative to the rest of the wall of the appendage 304 to allow for better transmission of the PD and LED of the oxygen sensor 360.

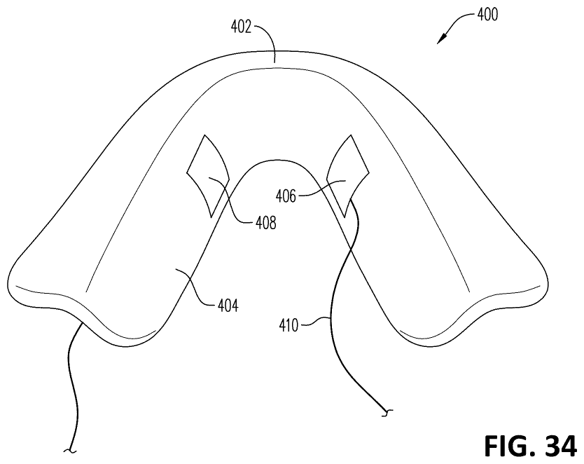

With reference to FIGS. 34-36, the present disclosure provides another embodiment of an oral appliance 400 for treating and/or collecting data used for treating sleep apnea. During sleep, electromyogram (EMG) can serve as an indicator of atonia (temporary paralysis of the user's legs and arms) while in REM vs non-REM sleep. It is contemplated that the oral appliance 400, which includes an EMG sensor, can provide data that would be indicative of respiratory events and arousals from apnea, and bruxism during sleep disturbances. EMG activity in subjects with sleep disorders may be typically more than twice as many as those without sleep disorders. It is contemplated that measuring the EMG of a user's intra-oral muscles may provide more sensitive and accurate data sets compared to external muscle EMG such as on the chin or the tibia.

The tongue is the largest and most active muscle in the oral cavity. It is also the upper respiratory muscle which is most involved in controlling air flow to the back of the throat during sleep. Hence, detecting its contractility during sleep can provide valuable physiological data for monitoring and diagnostic purposes. Because of the tongue's important role in respiration, detecting its electric activity during obstructive sleep apnea is valuable. The contemplated oral appliance 400 may be configured to detect such activities by placing EMG sensors along the right and left sides of the inferior-lateral aspects of the tongue while coupled to a removable mouthpiece. It is contemplated that the EMG sensor may partially or fully cover the anterior and posterior of the user's tongue, detecting the expiratory and inspiratory motor units associated with muscle fibers of the user's tongue.

As illustrated FIG. 34, an exemplary oral appliance 400 includes a mouthpiece 402 configured for being positioned in an oral cavity of a user. According to an aspect, the mouthpiece 402 includes a lingual wall 404 configured for being placed adjacent the user's tongue. The mouthpiece 402 may be made according to any of the methods described above and from any of the materials described above. The mouthpiece 402 is configured to be positioned on the user's lower jaw or upper jaw.

The oral appliance 400 further includes a pair of electromyogram (EMG) EMG sensors 406, 408 (e.g., the MYOWARE muscle sensor). The EMG sensors 406, 408 are configured to provide physiological data for monitoring and diagnosing obstructive sleep apnea. According to an aspect, the EMG sensors 406, 408 are configured to detect an electrical activity of the user's tongue. The electrical activity may be indicative of a muscle contraction of the user's tongue. In one embodiment, a stimulator may be positioned in a manner the same or similar to the EMG sensors. The EMG sensors 406, 408 are configured to be in contact with right and left sides of the user's tongue such that the EMG sensors 406, 408 detect twitches or contractions of the user's tongue bilaterally. The EMG sensors 406, 408 may be disposed on the mouthpiece 402 such that the EMG sensors 406, 408 contact the right and left sides of the inferior-lateral aspects of the user's tongue. It is contemplated that the EMG sensors 406, 408 may be sized so that they contact a partial area of the side(s) of the user's tongue. According to an aspect, the EMG sensors 406, 408 may be sized so that they can fully cover the anterior and posterior lateral surfaces of the user's tongue. It is contemplated that by being positioned to be in contact with the lateral surfaces of the user's tongue, the EMG sensors 406, 408 detect the expiratory and inspiratory motor units associated with muscle fibers of the user's tongue.

According to an aspect, the oral appliance 400 may include a single EMG sensor that contacts a single lateral surface of the user's tongue. Alternatively, multiple EMG sensors may be disposed on a single side of the mouthpiece 400 so that they all contact the same side (right of left lateral sides, for example) of the user's tongue. While FIG. 34 illustrates that the oral appliance 400 may include a wire 410 extending from the EMG sensors 406, 408, it is contemplated that the oral appliance 400 may be wireless.

According to an aspect, the oral appliance 400 may include a processor (not explicitly shown). The processor may include a micro-processor. According to an aspect, the EMG sensors 406, 408, in combination with the micro-processor, may be configured to identify respiratory events and arousals from obstructive sleep apnea. The EMG sensors 406, 408, in combination with the micro-processor, may be configured to identify bruxism during sleep disturbances.

The oral appliance 400 may further include a storage chip (not explicitly shown) configured to store and record data collected by the EMG sensors 406, 408. According to an aspect, a battery (not explicitly shown) may be provided to supply power to at least one of the EMG sensors 406, 408, the processor or micro-processor, and the storage chip. The battery may be a rechargeable battery. The oral appliance 400 may be positioned on a charger platform (not explicitly shown) so that the rechargeable battery can be charged so that it can supply power to the oral appliance 400.

When worn during sleep, the oral appliance 400 may provide information that serves as an indicator of at least one of atonia while the user is in REM sleep, respiratory events and arousals from apnea, and bruxism during sleep disturbances.

It is further contemplated that the oral appliance 400, in addition to the EMG sensor(s) 406, 408, may include an additional sensor (not explicitly shown). The additional sensor may be at least one of an oxygen sensor, a PPG sensor, an inertial movement sensor, or a sound measuring device. The EMG sensors 406, 408 and the additional sensor(s) may collectively gather physiologic data over time that aids in diagnosing obstructive sleep apnea. The micro-processor and rechargeable battery(s) may be sealed within the mouthpiece 400 or otherwise coupled to the mouthpiece 400.

Information or data gathered by the EMG sensors 406, 408 and the additional sensor(s) may be live streamed onto a smart device, for example, wirelessly via i.e., Bluetooth. The data can also be downloaded the next day by placing the appliance on a stationary hub/charger combination (not shown). The data can then be transferred onto a secure cloud based medium to be shared with the user as well as their care provider(s). The accumulation of this collective data may be built into a predictive model through deep thinking/artificial intelligence (AI) system integration to optimize the accurate diagnosis and subsequent monitoring of the ongoing treatment of patients suffering from sleep disordered breathing, i.e., obstructive sleep apnea. Additionally, the predictive model can assist in the determination of the most statistically efficacious prescribed therapy for OSA, with the highest probability of success, prior to the user engaging in any therapy which might provide sub-standard treatment response, such as positive air pressure (i.e., CPAP), custom oral sleep appliances, hypoglossal nerve stimulator, and pharmacotherapy.

It is contemplated that the EMG sensors 406, 408 may be suitably coupled to any of the oral appliances described herein.

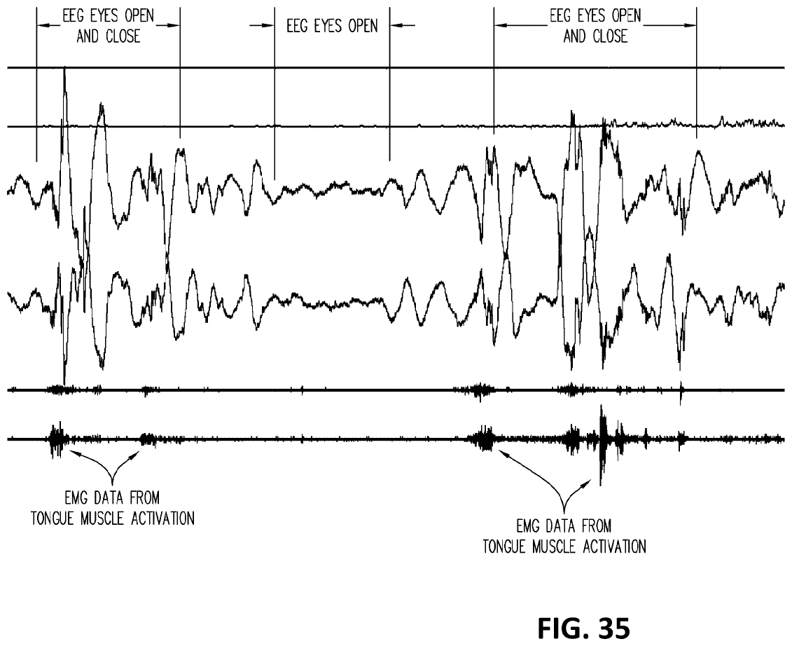

The additional sensor coupled to the oral appliance 400 may be an EEG sensor. With reference to FIG. 35, EMG data was obtained using an EEG sensor to determine when a user's eyes are opened or closed: (Phase 1) the user's eyes were opened and then closed; (Phase 2) when the user's eyes were open; and (Phase 3) again, when the user's eyes were opened and then closed. During that test period, EMG data was gathered during Phase 1 and Phase 3. Distinct signals were obtained that reflected the electrical activity in the user's tongue.

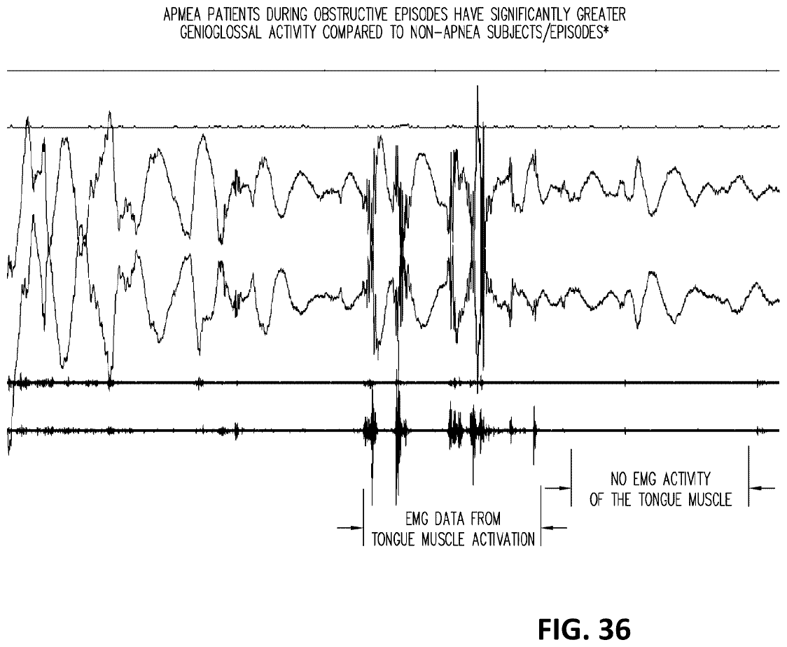

FIG. 36 is a chart that illustrates the difference between EMG data representing tongue contraction and EMG data representing an absence of tongue contraction. As illustrated, distinct signals are obtained that reflect when there is muscle contraction or twitching of the user's tongue, which indicates the user is not in REM sleep whereas signals reflecting tongue muscle relaxation indicates the user is in REM sleep. No distinct signals were created during the absence of muscle contraction or twitching of the user's tongue.

There are a number of example embodiments described herein.

Example 1 is an oral appliance, comprising: a mouthpiece configured to be positioned in an oral cavity of a user; an inertial measurement unit (IMU) coupled to the mouthpiece, the IMU configured to generate data based on movement of a user; a sound measuring device coupled to the mouthpiece, the sound measuring device configured to generate data based on noises of the user; and a processor coupled to the mouthpiece, the processor configured to detect a condition of the user based on analysis of the data generated by the IMU or the data generated by the sound measuring device.

Example 2 is the oral appliance of Example 1 that may optionally include a stimulator to provide electrical stimulation to a muscle in the oral cavity of the user based on detection of the condition of the user.

Example 3 is the oral appliance of Example 1 that may optionally include that the condition of the user includes at least one of normal breathing, abnormal breathing, awake periods, breath intensity, changes in breath sound intensity, snore sound quality, upper airway collapse, obstructive sleep apnea, sleep disordered breathing, total sleep time, sleep disturbances followed by arousal, jaw clenching, grinding, swallowing, a respiratory disease, or a neurodegenerative disorder.

Example 4 is the oral appliance of Example 1 that may optionally that the mouthpiece comprises an upper mouthpiece configured to be positioned on an upper jaw of the user and a lower mouthpiece configured to be positioned on a lower jaw of the user, and wherein the sound measuring device is coupled to the upper mouthpiece and the IMU is coupled to the lower mouthpiece.

Example 5 is the oral appliance of Example 4 that may optionally include that the IMU is coupled to an anterior portion of the lower mouthpiece.

Example 6 is the oral appliance of Example 4 that may optionally include that the sound measuring device is coupled to a posterior portion of the upper mouthpiece.

Example 7 is the oral appliance of Example 1 that may optionally include: a storage chip configured to store and record the data generated by the IMU or the data generated by the sound measuring device; and a battery configured to provide power to at least one of the IMU, the sound measuring device, the processor, or the storage chip.

Example 8 is the oral appliance of Example 1 that may optionally include that the mouthpiece comprises a buccal wall, a lingual wall, and an occlusal wall extending between the buccal wall and the lingual wall and the sound measuring device is secured to or embedded within the occlusal wall, such that the sound measuring device is adjacent to an upper second molar region of the user.

Example 9 is the oral appliance of Example 1 that may optionally include that the sound measuring device comprises a micro-electro-mechanical system (MEMS) microphone.

Example 10 is the oral appliance of Example 1 that may optionally include that the sound measuring device is disposed in a circular housing having a sealed first side and an open second side, wherein the sealed first side is secured to the mouthpiece by an adhesive and the sound measuring device is adjacent a suprasternal notch of the user to measure tracheal sound from the user.

Example 11 is the oral appliance of Example 1 that may optionally include that the IMU comprises at least one of a gyroscope and an accelerometer.

Example 12 is the oral appliance of Example 1 that may optionally include that the processor is configured to detect movement of an upper or lower jaw of the user based on the data generated by the IMU.

Example 13 is the oral appliance of Example 1 that may optionally include that the processor is configured to determine awake periods or arousal during sleep of the user based on detection of at least one of jaw clenching, grinding, or swallowing.

Example 14 is the oral appliance of Example 1 that may optionally include an amplifier configured to pre-amplify a signal from the sound measuring device, wherein the sound measuring device is configured to record sound at about 10 kHz.

Example 15 is a system comprising: an inertial measurement unit (IMU), the IMU configured to generate data based on movement of a user; a sound measuring device, the sound measuring device configured to generate data based on noises of the user; and a processor configured to detect a condition of the user based on analysis of the data generated by the IMU or the data generated by the sound measuring device.

Example 16 is the system of Example 15 that may optionally include a stimulator to provide electrical stimulation to a muscle in an oral cavity of a user based on detection of the condition of the user.

Example 17 is the system of Example 15 that may optionally include that the condition of the user includes at least one of normal breathing, abnormal breathing, breath intensity, changes in breath sound intensity, snore sound quality, upper airway collapse, obstructive sleep apnea, sleep disordered breathing, awake periods, total sleep time, sleep disturbances followed by arousal, jaw clenching, grinding, swallowing, a respiratory disease, or a neurodegenerative disorder.

Example 18 is the system of Example 15 that may optionally include: a storage chip configured to store and record the data generated by the IMU or the data generated by the sound measuring device; and a battery configured to provide power to at least one of the IMU, the sound measuring device, the processor, or the storage chip.

Example 19 is a method comprising: generating data, by an inertial measurement unit (IMU) based on movement of a user; generating data, by a sound measuring device, based on noises of the user; detecting, by a processor, a condition of the user based on analysis of the data generated by the IMU or the data generated by the sound measuring device; and providing electrical stimulation to a muscle in an oral cavity of the user based on detection of the condition of the user.

Example 20 is the method of Example 19 that may optionally include identifying a specific breathing pattern based on the data generated by the IMU or the data generated by the sound measuring device to detect the condition of the user.

Example 21 is an oral appliance comprising: a mouthpiece configured to be positioned in an oral cavity of a user; and an inertial measurement unit coupled to the mouthpiece.

Example 22 is the oral appliance of Example 21 that may optionally include that the inertial measurement unit comprises at least one of a gyroscope and an accelerometer.

Example 23 is the oral appliance of Example 21 that may optionally include that the mouthpiece is configured to be worn on the user's lower jaw.

Example 24 is the oral appliance of Example 23 that may optionally include that the inertial measurement unit is configured to detect movement of the user's upper or lower jaw.

Example 25 is the oral appliance of Example 23 that may optionally include that the inertial measurement unit is configured to detect at least one of snoring and awake periods or arousal during the user's sleep.

Example 26 is the oral appliance of Example 24 that may optionally include that the inertial measurement unit is configured to identify specific breathing patterns associated with sleep-disordered breathing via the detected movement of the user's lower jaw.

Example 27 is the oral appliance of any of Examples 21-26 that may optionally include a processor.

Example 28 is the oral appliance of Example 27 that may optionally include that the processor in combination with the inertial measurement unit is configured to identify the user's total sleep time, and total number of sleep disturbances followed by arousals during respiratory events.

Example 29 is the oral appliance of Example 27 that may optionally include that the processor in combination with the inertial measurement unit is configured to determine the user's jaw clenching and grinding, swallowing, thereby determining the user's awake periods or arousal during the user's sleep.

Example 30 is an oral appliance comprising: a mouthpiece configured to be positioned in an oral cavity of a user; and a sound measuring device coupled to the mouthpiece, wherein the sound measuring device is configured to detect at least one of the user's normal breathing, changes in the user's breath sound intensity, and the user's snore sound quality.

Example 31 is the oral appliance of Example 30 that may optionally include that the mouthpiece is configured to be positioned on the user's lower jaw or upper jaw.

Example 32 is the oral appliance of any of Examples 30 to 31 that may optionally include that the sound measuring device is a wired or wireless sound measuring device.

Example 33 is the oral appliance of any of Examples 30 to 32 that may optionally include that the sound measuring device is configured to detect upper airway collapse in instances of obstructive sleep apnea.

Example 34 is the oral appliance of any of Examples 30 to 33 that may optionally include that the sound measuring device is configured to identify specific breathing patterns associated with sleep-disordered breathing.

Example 35 is the oral appliance of Example 30 that may optionally include a processor.

Example 36 is the oral appliance of Example 35 that may optionally include that the sound measuring device in combination with the processor is configured to detect at least one of snoring and awake periods or arousal during the user's sleep.

Example 37 is the oral appliance of any of Examples 35 to 36 that may optionally include a storage chip configured to store and record data from the sound measuring device; and a battery configured to provide power to at least one of the sound measuring device, the processor, and the storage chip.

Example 38 is the oral appliance of any of Examples 35 to 37 that may optionally include that the processor in combination with the sound measuring device is configured to identify the user's total sleep time, and total number of sleep disturbances followed by arousals during respiratory events.

Example 39 is the oral appliance of any of Examples 35 to 38 that may optionally include a stimulator to provide electrical stimulation to a muscle in the user's oral cavity, wherein the sound measuring device is configured to detect changes in the user's air exchange intensity, and the processor is configured to translate the detected changes and trigger the stimulator to stimulate the muscle in the user's oral cavity to reverse the obstruction of the upper airway.

Example 40 is the oral appliance of any of Examples 30 to 39 that may optionally include that the sound measuring device is secured to or embedded within a posterior position of mouthpiece.

Example 41 is the oral appliance of any of Examples 30 to 40 that may optionally include that the sound measuring device is disposed in a circular housing having a sealed first side and an open second side, wherein the sealed first side is secured to the mouthpiece by an adhesive and the sound measuring device is adjacent the user's suprasternal notch to measure tracheal sound from the user.

Example 42 is the oral appliance of any of Examples 30 to 41 that may optionally include an amplifier configured to pre-amplify a signal from the sound measuring device, wherein the sound measuring device is configured to record sound at about 10 kHz.

Example 43 is the oral appliance of any of Examples 30 to 42 that may optionally include that the sound measuring device is configured to filter signals with: high pass τ=0.004 s [order 2]; and low pass τ==0.0005 Hz [order 2], wherein a root-mean-squared signal (moving median ⅙^{th}) is calculated to capture the magnitude of the sound.

Example 43 is the oral appliance of any of Examples 30 to 43 that may optionally include that the mouthpiece comprises a buccal wall, a lingual wall, and an occlusal wall extending between the buccal wall and the lingual wall.

Example 44 is the oral appliance of Example 43 that may optionally include that the sound measuring device is secured to or embedded within the occlusal wall, such that the sound measuring device is adjacent to the user's upper second molar region.

Example 45 is the oral appliance of any of Examples 30 to 44 that may optionally include that the sound measuring device comprises a microphone.

Example 46 is an oral noise monitor comprising: a mouthpiece configured to be positioned in an oral cavity of a user; a cannula embedded in the mouthpiece; and a sound measuring device adjacent the cannula, wherein the sound measuring device is configured to detect at least one of the user's normal breathing, changes in the user's breath sound intensity, and the user's snore sound quality.

Example 47 is the oral noise monitor of Example 46 that may optionally include that the mouthpiece comprises a first posterior end and a second posterior end, and the cannula extends between the first posterior end and the second posterior end.

Example 48 is the oral noise monitor of Example 46 that may optionally include that the cannula comprises a plurality of fenestrations, and the sound measuring device is adjacent the plurality of fenestrations.

Example 49 is the oral noise monitor of Example 46 that may optionally include that the sound measuring device comprises a noise sensor.

Example 50 is the oral noise monitor of Example 49 that may optionally include that the noise sensor comprises a MEMs mic.

The components of the apparatus illustrated are not limited to the specific embodiments described herein, but rather, features illustrated or described as part of one embodiment can be used on or in conjunction with other embodiments to yield yet a further embodiment. It is intended that the apparatus include such modifications and variations. Further, steps described in the method may be utilized independently and separately from other steps described herein.

While the apparatus and method have been described with reference to specific embodiments, it will be understood by those skilled in the art that various changes may be made and equivalents may be substituted for elements thereof without departing from the scope contemplated. In addition, many modifications may be made to adapt a particular situation or material to the teachings found herein without departing from the essential scope thereof.

In this specification and the claims that follow, reference will be made to a number of terms that have the following meanings. The singular forms “a,” “an” and “the” include plural referents unless the context clearly dictates otherwise. Furthermore, references to “one embodiment”, “some embodiments”, “an embodiment” and the like are not intended to be interpreted as excluding the existence of additional embodiments that also incorporate the recited features. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. In some instances, the approximating language may correspond to the precision of an instrument for measuring the value. Terms such as “first,” “second,” “upper,” “lower” etc. are used to identify one element from another, and unless otherwise specified are not meant to refer to a particular order or number of elements.

As used herein, the terms “may” and “may be” indicate a possibility of an occurrence within a set of circumstances; a possession of a specified property, characteristic or function; and/or qualify another verb by expressing one or more of an ability, capability, or possibility associated with the qualified verb. Accordingly, usage of “may” and “may be” indicates that a modified term is apparently appropriate, capable, or suitable for an indicated capacity, function, or usage, while taking into account that in some circumstances the modified term may sometimes not be appropriate, capable, or suitable. For example, in some circumstances an event or capacity can be expected, while in other circumstances the event or capacity cannot occur—this distinction is captured by the terms “may” and “may be.”

As used in the claims, the word “comprises” and its grammatical variants logically also subtend and include phrases of varying and differing extent such as for example, but not limited thereto, “consisting essentially of” and “consisting of” Where necessary, ranges have been supplied, and those ranges are inclusive of all sub-ranges therebetween. It is to be expected that variations in these ranges will suggest themselves to a practitioner having ordinary skill in the art and, where not already dedicated to the public, the appended claims should cover those variations.

Advances in science and technology may make equivalents and substitutions possible that are not now contemplated by reason of the imprecision of language; these variations should be covered by the appended claims. This written description uses examples to disclose the method, machine and computer-readable medium, including the best mode, and also to enable any person of ordinary skill in the art to practice these, including making and using any devices or systems and performing any incorporated methods. The patentable scope thereof is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal language of the claims.

What is claimed is:

1. An oral appliance, comprising:
a mouthpiece configured to be positioned in an oral cavity of a user;
an inertial measurement unit (IMU) coupled to the mouthpiece, the IMU configured to generate data based on movement of a user;
a sound measuring device coupled to the mouthpiece, the sound measuring device configured to generate data based on noises of the user; and
a processor coupled to the mouthpiece, the processor configured to detect a condition of the user based on analysis of the data generated by the IMU or the data generated by the sound measuring device.

2. The oral appliance of claim 1, further comprising a stimulator to provide electrical stimulation to a muscle in the oral cavity of the user based on detection of the condition of the user.

3. The oral appliance of claim 1, wherein the condition of the user includes at least one of normal breathing, abnormal breathing, awake periods, breath intensity, changes in breath sound intensity, snore sound quality, upper airway collapse, obstructive sleep apnea, sleep disordered breathing, total sleep time, sleep disturbances followed by arousal, jaw clenching, grinding, swallowing, a respiratory disease, or a neurodegenerative disorder.

4. The oral appliance of claim 1, wherein the mouthpiece comprises an upper mouthpiece configured to be positioned on an upper jaw of the user and a lower mouthpiece configured to be positioned on a lower jaw of the user, and wherein the sound measuring device is coupled to the upper mouthpiece and the IMU is coupled to the lower mouthpiece.

5. The oral appliance of claim 4, wherein the IMU is coupled to an anterior portion of the lower mouthpiece.

6. The oral appliance of claim 4, wherein the sound measuring device is coupled to a posterior portion of the upper mouthpiece.

7. The oral appliance of claim 1, further comprising:
a storage chip configured to store and record the data generated by the IMU or the data generated by the sound measuring device; and
a battery configured to provide power to at least one of the IMU, the sound measuring device, the processor, or the storage chip.

8. The oral appliance of claim 1, wherein the mouthpiece comprises a buccal wall, a lingual wall, and an occlusal wall extending between the buccal wall and the lingual wall and the sound measuring device is secured to or embedded within the occlusal wall, such that the sound measuring device is adjacent to an upper second molar region of the user.

9. The oral appliance of claim 1, wherein the sound measuring device comprises a micro-electro-mechanical system (MEMS) microphone.

10. The oral appliance of claim 1, wherein the sound measuring device is disposed in a circular housing having a sealed first side and an open second side, wherein the sealed first side is secured to the mouthpiece by an adhesive and the sound measuring device is adjacent A suprasternal notch of the user to measure tracheal sound from the user.

11. The oral appliance of claim 1, wherein the IMU comprises at least one of a gyroscope and an accelerometer.

12. The oral appliance of claim 1, wherein the processor is configured to detect movement of an upper or lower jaw of the user based on the data generated by the IMU.

13. The oral appliance of claim 1, wherein the processor is configured to determine awake periods or arousal during sleep of the user based on detection of at least one of jaw clenching, grinding, or swallowing.

14. The oral appliance of claim 1, further comprising an amplifier configured to pre-amplify a signal from the sound measuring device, wherein the sound measuring device is configured to record sound at about 10 kHz.

15. A system comprising:
an inertial measurement unit (IMU), the IMU configured to generate data based on movement of a user;
a sound measuring device, the sound measuring device configured to generate data based on noises of the user; and
a processor configured to detect a condition of the user based on analysis of the data generated by the IMU or the data generated by the sound measuring device.

16. The system of claim 15, further comprising a stimulator to provide electrical stimulation to a muscle in an oral cavity of a user based on detection of the condition of the user.

17. The system of claim 15, wherein the condition of the user includes at least one of normal breathing, abnormal breathing, breath intensity, changes in breath sound intensity, snore sound quality, upper airway collapse, obstructive sleep apnea, sleep disordered breathing, awake periods, total sleep time, sleep disturbances followed by arousal, jaw clenching, grinding, swallowing, a respiratory disease, or a neurodegenerative disorder.

18. The system of claim 15, further comprising:
a storage chip configured to store and record the data generated by the IMU or the data generated by the sound measuring device; and
a battery configured to provide power to at least one of the IMU, the sound measuring device, the processor, or the storage chip.

19. A method comprising:
generating data, by an inertial measurement unit (IMU) based on movement of a user;
generating data, by a sound measuring device, based on noises of the user;
detecting, by a processor, a condition of the user based on analysis of the data generated by the IMU or the data generated by the sound measuring device; and
providing electrical stimulation to a muscle in an oral cavity of the user based on detection of the condition of the user.

20. The method of claim 19, further comprising identifying a specific breathing pattern based on the data generated by the IMU or the data generated by the sound measuring device to detect the condition of the user.
